(19) United States
(12) Patent Application Publication
(10) Pub. No.: US 2026/0337366 A1
(43) Pub. Date: Oct. 29, 2026

(54) PROCESS FOR RECYCLING POLY(ALKYLENE TEREPHTHALATE)

(71) Applicant: Eastman Chemical Company, Kingsport, TN (US)

(72) Inventors: Neil Warren Boaz, Kingsport, TN (US); Jennifer Lloyd Stapleton, Mount Carmel, TN (US)

(73) Assignee: Eastman Chemical Company, Kingsport, TN (US)

(21) Appl. No.: 19/121,263

(22) PCT Filed: Oct. 24, 2023
(86) PCT No.: PCT/US2023/077573

§ 371 Date: Apr. 15, 2025

Related U.S. Application Data
(60) Provisional application No. 63/419,044, filed on Oct. 25, 2022.

(51) Int. Cl. C08J 11/24; C07C 67/03; C07C 67/54; C07C 67/56; C08J 11/28

(52) U.S. Cl. CPC C08J 11/24; C07C 67/03; C07C 67/54; C07C 67/56; C08J 11/28; C08J 2367/02

(57) ABSTRACT

Provided is a facile process for preparing dimethyl terephthalate, the process comprising treating a polyester stream with a C4-C14 alkanol in the presence of an esterification or transesterification catalyst in order to depolymerize the polyester. Cooling of this mixture allows removal of insoluble materials, followed by transesterification to afford recycled dimethyl terephthalate (r-DMT) and recycled ethylene glycol (r-EG).

### FIELD OF THE INVENTION

This invention relates to the field of polyester recycling, in particular the recycling of poly(ethylene terephthalate) (PET).

### BACKGROUND OF THE INVENTION

Recycling of plastics has become an important issue facing society. PET, which is a type of polyester, is one of the most widely recycled plastics, with most of the recycling being mechanical in nature, wherein the polyester is physically separated from other plastics, cleaned, and re-processed into recycled PET (r-PET). Mechanical recycling has limited utility, as each heat-up cycle results in some degradation of the PET. An alternative to mechanical recycling is chemical recycling, wherein the polyester is chemically broken down into constituent monomers. This allows purification of these monomers followed by re-polymerization into PET or other polyesters affording polymer identical to virgin material.

Chemical recycling may be categorized by the depolymerization agent used. For instance, depolymerization agents may include water, methanol, and ethylene glycol. If the depolymerization reactant is water, the products are terephthalic acid and ethylene glycol. If it is methanol, the products are dimethyl terephthalate and ethylene glycol. If it is ethylene glycol, the product is bis(hydroxyethyl) terephthalate (BHET) or oligomers thereof (depending on how much ethylene glycol is used). The key issue for the success of polyester chemical recycling is the ability to economically generate the monomers in sufficient purity for re-polymerization. This can pose different degrees of challenge depending upon the monomer target chosen.

Terephthalic acid may be used as a monomer, as it is the most widely used species for preparing polyesters such as PET. Neutral or acid-catalyzed hydrolysis has poor kinetics and requires very forcing conditions. Thus, caustic hydrolysis is often used. However, base hydrolysis generates disodium terephthalate, which must be protonated to afford the desired acid. The protonation generates two equivalents of a salt such as sodium chloride per mole of terephthalic acid, and this salt must either be disposed of or recycled which causes additional expense. In addition, purification of the terephthalic acid to a quality sufficient for polymer preparation can be challenging.

Bis(hydroxyethyl) terephthalate (BHET) is an attractive alternative depolymerization monomer target, particularly for PET preparation, as it is the actual monomer that is polymerized to PET. Glycolysis of PET to BHET has been broadly investigated, and recent advances include technologies such as volatile amine catalysts, magnetic ionic liquid catalysts, and microwave technologies. Although BHET can be used for chemical recycling, purification of BHET to polymer-grade purity is challenging, and often involves resource-intensive multi-stage processing.

Dimethyl terephthalate (DMT) is often not the most desirable recycling monomer target for many polymer manufacturers, as these manufacturers are not configured to deal with the methanol by-product released upon re-polymerization. However, if a facility is engineered to handle methanol, DMT can be an attractive target, as this molecule is relatively simple to purify. Methanolysis of PET has received significant attention in the past. The main issue with direct methanolysis of polyesters, including PET, is the high temperatures required for sufficient polyester reactivity. These temperatures are significantly higher than the boiling point of methanol, and either require high pressure (often including the use of supercritical methanol) or the use of superheated methanol vapor to effect the methanolysis.

Thus, improved methodologies for depolymerization of PET and isolation of recycle content DMT (r-DMT) would be of great interest.

### SUMMARY OF THE INVENTION

Irrespective of the methodology used in chemical recycling, a particular issue in depolymerization of waste PET is the removal of non-polyester impurities. These are often insoluble solid materials that are unrelated to polyester, and thus are most effectively removed via filtration. Unfortunately, direct methanolysis affords DMT as a solid (melting point 145 °C), so filtration of the mixture requires high temperatures. In addition, although BHET is lower melting (106 °C), glycolysis often generates significant amounts of BHET dimers and trimers (melting points >200 °C) unless an undesirably large excess of ethylene glycol is used (which results in increased reactor volumes and EG recycle loops), so filtration of these often-viscous mixtures also requires significantly elevated temperatures. In addition to the high temperature high viscosity processing challenges, the elevated temperatures necessary for these filtrations can tend to soften and even liquify some of the impurities in the waste PET feedstocks, and thus their removal can be difficult.

In various aspects as set forth herein, the invention provides the generation of a depolymerization mixture that is a low viscosity liquid at low temperature (below 100 °C), which allows simple and early removal of these impurities by solid-liquid separation (SLS) such that they do not impact further processing. Solid-liquid separation refers to multiple technologies known to those in the art including decantation, filtration, centrifugation, etc. Additionally, the invention also provides a process which incorporates high concentrations, relatively low temperatures, simple recycle loops and results in r-DMT and recycled ethylene glycol (r-EG) with high purity.

### DETAILED DESCRIPTION OF THE INVENTION

The present invention provides a process for recycling polyesters which involves two chemical steps: (1) depolymerization of a polyester with a higher carbon monohydric alcohol, followed by (2) conversion of the depolymerization mixture to recycled DMT (r-DMT) and recycled ethylene glycol (r-EG). In other words, the process involves the depolymerization of polyesters by a catalyzed reaction (with an esterification or transesterification catalyst) with a higher alcohol, i.e., a C₄-C₁₄ alcohol, removal of solid impurities via SLS, and conversion of the depolymerized mixture to afford r-DMT and r-EG. The process of the invention thus has the advantage of relatively low depolymerization temperature (and hence lower energy usage), high yields, and high r-DMT purity. In particular, the purity of the resulting r-DMT can reduce or eliminate the need for further purification of the r-DMT so produced.

Thus, in a first aspect, the invention provides a process for preparing dimethyl terephthalate, the process comprising:
a. treating a poly(C₂-C₄ alkylene) terephthalate with a C₄-C₁₄ alkanol in the presence of an esterification or transesterification catalyst at a temperature sufficient to depolymerize at least a portion of the poly(C₂-C₄ alkylene terephthalate), to afford a first reaction mixture product comprising a di-C₄-C₁₄ alkyl terephthalate; followed by
b. treatment with a transesterification catalyst in the presence of methanol, to provide a second reaction mixture product comprising dimethyl terephthalate, a C₄-C₁₄ alcohol, and ethylene glycol, followed by
c. isolation of the dimethyl terephthalate.

In certain embodiments, the poly(C₂-C₄)alkylene terephthalate may be further comprised of additional glycol and/or diacid residues, and include, for example, polyethylene terephthalate (PET), 1,4-cyclohexanedimethanol (CHDM)-modified PET, isophthalic acid (IPA)-modified PET, diethylene glycol (DEG)-modified PET, glycol-modified PET, neopentyl glycol (NPG)-modified PET, propane diol (PDO)-modified PET, butanediol (BDO)-modified PET, hexanediol (HDO)-modified PET, 2-methyl-2,4-pentanediol (MP diol)-modified PET, isosorbide-modified PET, poly(tetramethylene ether) glycol (PTMG)-modified PET, poly(ethylene) glycol (PEG)-modified PET, polycyclohexylenedimethylene terephthalate (PCT), cyclohexanedimethanol (CHDM)-containing copolyester, isosorbide-containing copolyester, or a combination thereof. In other embodiments, the poly(C₂-C₄)alkylene terephthalate can include polyethylene terephthalate (PET) that comprises residues of CHDM, IPA, DEG, NPG, PDO, BDO, HDO, MP diol, isosorbide, PTMG, PEG, or a combination thereof.

In certain embodiments, the esterification or transesterification catalyst for part a. is chosen from such known catalysts, for example Lewis Acids and Lewis Bases. Exemplary Lewis Base catalysts include alkali metal alkoxides, alkali metal carbonates, or alkali metal bicarbonates, wherein the alkali metals are chosen from lithium, sodium, potassium, or cesium. Exemplary Lewis Acid catalysts include metal acetates, titanium alkoxides, and tin species such as tin oxalate, monobutyltin oxide, and monobutyltin tris(2-ethylhexanoate). In certain embodiments, the metal acetates are chosen from alkali metal acetates and transition metal acetates. In certain embodiments, the catalysts referred to above include potassium carbonate, potassium bicarbonate, potassium acetate, titanium tetra(isopropoxide and monobutyltin tris(2-ethylhexanoate). In certain embodiments, the amount of catalyst utilized in the depolymerization reaction (step a. above) is from about 0.001 to about 0.1 equivalents based on a PET repeat unit, or between about 0.005 and about 0.075 equivalents, or between about 0.005 and about 0.05 equivalents.

In step a. above, the temperature is in certain embodiments, between about 100 °C to about 250 °C, about 125°to about 250 °C, or about 150 °C to about 220 °C. The temperature may be advantageously chosen to afford desired reaction rates while minimizing reaction by-products, and while utilizing simplicity in equipment set-up. In this regard, it may be convenient to run the process at the boiling point of the lowest-boiling component of the mixture at ambient pressure. In other cases, for example with the lower carbon alcohols, it may be more convenient to run the process under elevated pressure in order to elevate the boiling point of the mixture and to accelerate the rate of reaction. In certain embodiments, the reaction pressure may range from about 0 to about 500 psig (pounds per square inch gauge).

In general, the time required for step a. to be completed is dependent on the temperature, the particular monohydric alcohol chosen, and the amount of catalyst utilized. In certain embodiments, the reaction time is between about 0.5 and about 10 hours, or between about 1 and about 8 hours, or between about 1 and about 6 hours.

Advantageously, the low-viscosity depolymerized product mixture resulting from step a. can be cooled to between about 20° and about 100 °C prior to SLS. Cooling facilitates the separation by both ensuring that the impurities have a chance to fully solidify as well as using standard equipment rather than high temperature SLS equipment. Thus, in certain embodiments, the temperature prior to SLS is between about 20 °C and about 100 °C, or between about 30 °C and about 80 °C, or between about 40 °C and about 70 °C SLS can be carried out using standard techniques known in the art such as vacuum filtration, centrifugation, or pressure filtration. There may be cases where filter aid is desirable as is known in the art. In some cases, it may be more desirable to decant the liquid phase rather than filter the mixture. As used herein, the term “isolating” or “isolation” refers to known methods of segregating solid and liquid materials, such as the SLS methods described above.

A second step (b.) comprises conversion of the higher alcohol depolymerized mixture to dimethyl terephthalate (DMT) and ethylene glycol (EG) using a base-catalyzed low temperature transesterification with methanol. In certain embodiments, this step comprises mixing the depolymerized mixture with methanol; adding a transesterification catalyst at an initial temperature of between about 40 °C and about 70 °C; holding at that initial temperature for an initial period of time; cooling over a second period of time to a final temperature of about 30 °C or below; holding at that final temperature for a third period of time to afford a final DMT slurry; and isolating the DMT, for example by solid-liquid separation such as filtration.

The amount of methanol used in this step of the process is not crucial but is generally a sufficient amount to maintain a fluid slurry of solid DMT in the mixture at the final temperature. In general, the amount of methanol can be between about 5 and about 50 equivalents based on the starting polyester substrate, or between about 5 and about 36 equivalents, or between about 5 and about 24 equivalents.

In certain embodiments, basic catalysts are utilized for this transesterification step of the process. Exemplary catalysts include metal C₁-C₁₄ alkoxides, metal carbonates, and metal hydroxides. In certain embodiments, the metals are alkali metals such as lithium, sodium, and potassium. In other embodiments, the catalysts are chosen from sodium or potassium hydroxide, sodium or potassium methoxide, sodium or potassium 2-ethylhexylate, or potassium carbonate. The amount of catalyst can in certain embodiments be from about 0.005 to about 0.2 equivalents, based on the starting polyester substrate, or between about 0.01 and about 0.10 equivalents, or between about 0.02 and about 0.075 equivalents, with more catalyst generally affording a faster reaction.

The initial temperature for this transesterification step of the process is, in certain embodiments, between about 40 °C and 70 °C, or between about 40 °C and about 65 °C. In one embodiment, the initial temperature is between about 50 °C and about 65 °C.

In certain embodiments, the first period of time at the initial temperature is about 15 to about 120 minutes, or about 15 to about 90 min, or about 15 to about 60 minutes. The cooling time is not particularly crucial, and generally can occur over 5 to 75 minutes, or 15 to 60 minutes, or 15 to 45 minutes. The time at the final temperature is not crucial, and can be from 0 to 24 hours, or 0 to 120 minutes, or 0 to 60 minutes. These times can be modified to a certain extent by catalyst loading.

This second step (step b.) of the process (i.e., transesterification) can be operated at or near atmospheric pressure. Conditions above atmospheric pressure, although not required, would not have a negative effect on the process. Conditions below atmospheric pressure during the process could result in the evaporation of methanol, which would be undesirable, but could also assist in cooling the mixture if the evaporation of methanol is well controlled.

The conditions used above result in a slurry of DMT in methanol, and it is convenient and effective to isolate the DMT by solid-liquid separation. The method of SLS can be any conventional method known in the art—vacuum filtration, centrifugation, pressure filtration, etc. Additional methanol is often used to transfer the solid DMT to the filter, and this methanol also helps remove any residual glycol and monohydric alcohol from the solid. The DMT thus obtained from this process is highly pure with generally, e.g., <0.5% non-volatile impurities with an overall single pass yield from PET often over 80%.

Another embodiment of the process involves separation of the liquid components after isolation of the DMT. These components are generally methanol, the monohydric higher alcohol, ethylene glycol, and residual soluble terephthalate species. One embodiment of this separation process involves sequential distillation of the liquid components in the resulting filtrate, thereby purifying same and allowing for recycle of solvents and feedstocks in to the process. This is particularly advantaged for the shorter monohydric alcohols such as n-butanol, i-butanol, and hexanol, as the boiling points of the liquid species are well separated. This sequential distillation allows recovery of methanol, the alcohol, and ethylene glycol in high purity, respectively. In particular, the ethylene glycol from this distillation has low color. The residual terephthalates end up in the distillation pot residue, and thus can be recycled into the process.

Another embodiment of this separation process is effective for high-boiling monohydric alcohols such as decanol, dodecanol and tetradecanol. In this embodiment the methanol and ethylene glycol can be isolated by sequential distillation while the monohydric alcohol remains in the base with the terephthalate residues, which allows the ready recycle of this latter mixture back into the process.

Another embodiment of the separation process involves distillation and extraction and is of particular use for the mid-range monohydric alcohols where the boiling point of the alcohol is close to that of ethylene glycol (e.g., 2-ethylhexanol, n-octanol). In this embodiment the methanol is removed by distillation and the residue is treated with water. The amount of water is not crucial and can vary from 0.1 to 5 parts per part of residue, or 0.1 to 1 part, or 0.25 to 0.75 part per part of residue. This water treatment affords two layers, which can be readily separated. The top layer is nearly pure monohydric alcohol, which contains most of the terephthalate residues; this material can be recycled into the process. The bottom layer contains water and ethylene glycol with a small amount of the monohydric alcohol. This residual monohydric alcohol can be removed as a water azeotrope during water distillation. The overall recovery of the monohydric alcohol is generally above 95%. Further distillation affords highly pure colorless EG in nearly quantitative yield from the DMT filtrate.

Further advantages of the invention include the ability to utilize plastic waste streams obtained by conventional separation or mechanical recycling of various plastics. In this regard, the robust process described herein enables the separation of such undesired materials from the desired recycle materials.

As used herein, the term “waste plastic stream” refers to a heterogeneous waste stream comprising various polymers and plastics. The waste plastic stream may include material that was recovered as manufacturing scrap, industrial waste, post-consumer waste, or a combination thereof. In certain embodiments, the recycled polyester(s) can be prior-used products that have been used and/or discarded. In certain embodiments, the waste plastic stream can come from various sources and/or in various forms, including but not limited to textiles, carpet, thermoformed materials, bottles, pellets, and film. Additionally, the waste plastic stream can include one or more foreign materials. In certain embodiments, the one or more foreign materials may include, but are not limited to, polyesters other than poly(ethylene terephthalate), polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, or a combination thereof. In various aspects, the one or more foreign materials can be present in the polyester composition in an amount of from about 0.01 wt. % to about 50 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 7.5 wt. %, about 0.01 wt. % to about 5 wt. %, about 0.01 wt. % to about 2.5 wt. %, about 0.01 wt. % to about 1.0 wt. %, relative to the weight of the poly(C 2-C4)alkylene terephthalate.

Thus, a waste plastic stream is generally obtained by a recycling process which involves collection and optionally segregation of the plastic materials by polymer type, often followed by forming same into a molten waste plastic and into a new intermediate product (e.g., pellets or sheets) and/or a new end product. Generally, mechanical recycling does not substantially change the chemical structure of the plastic being recycled.

Thus, in a second aspect, the invention provides a process for preparing dimethyl terephthalate, the process comprising:
a. treating a waste plastic stream comprising poly(ethylene terephthalate) with a C₄-C₁₄ alkanol in the presence of an esterification or transesterification catalyst at a temperature sufficient to depolymerize at least a portion of the poly(ethylene terephthalate), to afford a first reaction mixture product comprising a di-C₄-C₁₄ alkyl terephthalate; followed by
b. treatment with a transesterification catalyst in the presence of methanol, to provide a second reaction mixture product comprising dimethyl terephthalate, a C₄-C₁₄ alcohol, and ethylene glycol, followed by
c. isolation of the dimethyl terephthalate.

This second aspect of the invention can also utilize the various embodiments as set forth above in the first aspect.

Key advantages of the process of the invention include the ability to depolymerize polyester with higher monohydric alcohols at low alcohol to PET ratios and still afford a non-viscous depolymerization mixture that can undergo SLS at low temperature, thereby effectively removing solid inorganic contaminants originally found in the waste plastic stream along with non-PET plastics such as those described above. In addition, the process runs at relatively low overall temperatures with low energy usage using inexpensive catalysts with short reaction times. In general, the r-DMT generated by this method is often obtained in 80% yield or greater from PET with <0.5% of non-volatile impurities. Indeed, even closely related impurities such as dimethyl isophthalate are removed from the dimethyl terephthalate by this invention. This invention can allow the direct production of DMT that is of sufficient purity (or with minimal subsequent purification) to allow its direct use in polyester production. In addition, the monohydric alcohol reactant is readily recycled with high overall recovery and the r-EG is obtained in high purity and high yield with good sensorics (low color and odor).

### EXAMPLES

### Example 1: Depolymerization of PET with n-butanol at a 2.5:1 Ratio

Pelletized bottle-flake waste PET (50.0 g; 0.276 mol) and potassium carbonate (1.01 g; 0.0072 mol; 0.028 equivalents) were slurried in n-butanol (125 g; 1.686 mol; 6.55 equivalents) in a 300-mL autoclave. The autoclave was pressurized with 500 psig nitrogen, then vented and sealed. Agitation was started and the autoclave was heated to 200 °C internal to generate about 120 psig autologous pressure. The vessel was further pressurized to 500 psig and stirred at 200 °C for four hours. The mixture was cooled to ambient temperature and poured out of the autoclave into a bottle (little remained in the autoclave). The non-viscous mixture was filtered to remove insolubles, and the filtrate was bottled. The insoluble residue was dried to afford 0.60 g. HPLC analysis of the filtrate indicated 71% dibutyl terephthalate and the material analyzed at an acid number of 1.67. The theoretical content of the filtrate was 1.46 mmol total terephthalate per gram. HPLC (150×4.6 mm ZORBAX SB-C8 column, 85:15 (v:v) methanol: water (containing 0.1% trifluoroacetic acid) for 6 min, gradient to 100% methanol over 1 min, 100% methanol for 3 minutes, 220 nm detection): dibutyl terephthalate, t_{R} 4.53 minutes; butyl EG mixed ester, t_{R} 2.25 minutes.

### Example 2: Depolymerization of PET With 2:1 Ratio

Pelletized bottle-flake waste PET (50.0 g; 0.276 mol) and potassium carbonate (1.01 g; 0.0072 mol; 0.028 equivalents) were slurried in n-butanol (100 g; 1.349 mol; 5.24 equiv) in a 300-mL autoclave. The autoclave was pressurized with 500 psig nitrogen, then vented and sealed. Agitation was started and the autoclave was heated to 200 °C internal to generate about 120 psig autologous pressure. The vessel was further pressurized to 500 psig and stirred at 200 °C for four hours. The mixture was cooled to ambient temperature and poured out of the autoclave into a bottle (little remained in the autoclave). The non-viscous mixture was filtered to remove insolubles, and the filtrate was bottled. The insoluble residue was dried to afford <1 g. HPLC analysis of the filtrate indicated 70% dibutyl terephthalate and the material analyzed at an acid number of 1.58. The theoretical content of the filtrate was 1.72 mmol total terephthalate per gram.

### Example 3: Transesterification of PET Butanolysis Mixture With methanol Using Sodium Methoxide

PET butanolysis mixture prepared as in Example 2 (1.72 mmol/g; 40.0 g; 0.069 mol) was slurried in 50.2 mL of methanol (1.238 mol; 18.0 equiv) in a 300-mL 3-neck round-bottom flask with an overhead stirrer, a thermocouple, and an air condenser with a nitrogen inlet. The flask was placed into an oil bath set at 57°C, and once the reaction mixture equilibrated at 50 °C (+/−2 °C) 25% sodium methoxide in methanol (787 μL; 0.0034 mol; 0.05 equivalents; 0.34 equivalents active catalyst based on acid number) was added. The mixture was stirred at 250 rpm at 50 °C and precipitation with an attendant exotherm was noted starting at 4.5 minutes. After heating for 60 minutes at 50 °C the heating bath was then removed, and the mixture was allowed to cool to ambient temperature over 45 minutes and then stirred at ambient temperature for one hour. The resulting precipitate was filtered, washed with methanol, and air-dried to afford 9.70 g of DMT as a white powder which assayed at >99.9 weight % purity by HPLC, indicating 73% overall yield from PET. Analysis of the filtrate indicated that it contained 5.5% of the expected yield of DMT and small amounts (<0.5% each) of bis(hydroxyethyl) terephthalate (BHET), methyl hydroxyethyl terephthalate (MHET), and dimethyl isophthalate (DMI).
¹H NMR (CDCI₃): δ8.11 (s, 4H); 3.96 (s, 3H).

HPLC (150×4.6 mm Zorbax SB-C8 column, 75:25 (v:v) methanol: water (containing 0.1% trifluoroacetic acid) for 5 min, gradient to 100% methanol over 1 min, hold at 100% methanol for 4 min, 220 nm detection): BHET, t_{R} 1.75 min. ; MHET, t_{R} 2.1 min. ; DMI, t_{R} 2.75 min. ; DMT, t_{R} 2.87 min.

### Example 4: Transesterification of Pet Butanolysis Mixture With Methanol Using Sodium Hydroxide

PET butanolysis mixture prepared as in Example 2 (1.72 mmol/g; 40.0 g; 0.069 mol) was slurried in 50.2 mL of methanol (1.238 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/31 2°C) 50% aqueous sodium hydroxide (168 μL; 0.255 g; 0.0032 mol; 0.046 equivalents; 0.030 equivalents active catalyst based on acid number) was added. The mixture was stirred at 250 rpm at 50 °C and precipitation with an attendant exotherm was noted starting at five minutes. After heating for 60 minutes at 50 °C the heating bath was then removed, and the mixture was allowed to cool to ambient temperature over 45 minutes and then stirred at ambient temperature for one hour. The resulting precipitate was filtered, washed with methanol, and air-dried to afford 9.70 g of DMT as a white powder which assayed at >99.9 weight % purity by HPLC, indicating 66% overall yield from PET. Analysis of the filtrate indicated that it contained 5.5% of the expected yield of DMT and small amounts (<0.5% each) of bis(hydroxyethyl) terephthalate (BHET), methyl hydroxyethyl terephthalate (MHET), and dimethyl isophthalate (DMI).

### Example 5: Depolymerization of Pet With N-butanol at a 1.5:1 Ratio

Pelletized bottle-flake waste PET (250.0 g; 1.287 mol) and potassium carbonate (4.98 g; 0.036 mol; 0.028 equivalents) were slurried in n-butanol (375 g; 5.06 mol; 3.93 equivalents) in a 1-L autoclave. The autoclave was pressurized with 500 psig nitrogen, then vented and sealed. Agitation was started and the autoclave was heated to 200 °C internal to generate about 120 psig autologous pressure. The vessel was further pressurized to 500 psig and stirred at 200 °C for four hours. The mixture was cooled to ambient temperature and poured out of the autoclave into a bottle (little remained in the autoclave). The non-viscous mixture was filtered to remove insolubles. The viscosity of the filtrate was analyzed and indicated a viscosity that decreased from 8.6 cP at 25 °C to 4.2 cP at 60 °C. HPLC analysis of the filtrate indicated 59% dibutyl terephthalate and the material analyzed at an acid number of 1.36. The theoretical content of the filtrate was 2.06 mmol total terephthalate per gram.

### Example 6: Transesterification of PET Butanolysis Mixture With Methanol Using Sodium Methoxide

PET butanolysis mixture prepared as in Example 5 (2.06 mmol/g; 35.0 g; 0.072 mol) was slurried in 61.5 mL of methanol (1.518 mol; 21.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (632 μL; 0.0028 mol; 0.038 equivalents; 0.30 equivalents active catalyst based on acid number) was added. The mixture was stirred at 250 rpm at 50 °C and precipitation with an attendant exotherm was noted starting at five minutes. After heating for 60 minutes at 50 °C the heating bath was then removed, and the mixture was allowed to cool to ambient temperature over 45 minutes and then stirred at ambient temperature for two hours. The resulting precipitate was filtered, washed with methanol, and air-dried to afford 11.78 g of DMT as a white powder which assayed at >99.9 weight % purity by HPLC, indicating 84% overall yield from PET. Analysis of the filtrate indicated that it contained 3.3% of the expected yield of DMT and small amounts (<0.5% each) of bis(hydroxyethyl) terephthalate (BHET), methyl hydroxyethyl terephthalate (MHET), and dimethyl isophthalate (DMI).

### Example 7: Transesterification of Pet Butanolysis Mixture With Methanol Using Sodium Hydroxide

PET butanolysis mixture prepared as in Example 5 (2.06 mmol/g; 150.0 g; 0.309 mol) was slurried in 264 mL of methanol (6.504 mol; 21.0 equivalents) in a 1 L jacketed reactor with an overhead stirrer, a thermocouple, and an air condenser with a nitrogen inlet. The jacket was heated until the internal temperature had equilibrated at 50 °C (+/−2 °C). 50% Aqueous sodium hydroxide (688 μL; 0.0129 mol; 0.042 equivalents; 0.30 equivalents active catalyst based on acid number) was added. The mixture was stirred at 200 rpm at 50 °C and precipitation with an attendant exotherm was noted starting at 6.5 minutes. After heating for 60 minutes at 50 °C heating was discontinued and the mixture was allowed to cool to ambient temperature over 90 minutes and then stirred at ambient temperature for one hour. The resulting precipitate was filtered, washed with methanol, and air-dried to afford 49.17 g of DMT as a white powder which assayed at 99.7 weight % purity by HPLC, indicating 82% overall yield from PET. Analysis of the filtrate indicated that it contained 3.3% of the expected yield of DMT and small amounts (<0.5% each) of bis(hydroxyethyl) terephthalate (BHET), methyl hydroxyethyl terephthalate (MHET), and dimethyl isophthalate (DMI).

The resulting filtrate (407.57 g) was concentrated at reduced pressure to remove the bulk of the methanol to afford a slurry, which was filtered. The precipitate (1.58 g after drying) was analyzed at >99.8% DMT (2.6% yield). The liquid was distilled at atmospheric pressure through a Vigreux column to afford 84.03 g of distillate with boiling point of 62-80 °C. This distillate analyzed at 80.6% methanol and 19.4% butanol. The remainder was further distilled at atmospheric pressure to afford an overlap cut (8.88 g, boiling point 80-110 °C) and a fraction that boiled at 110-11 3°C (58.1 g), which analyzed at 98.5% butanol, 1.3% methanol, and 0.2% EG. The pressure was reduced to 30 mm Hg and an overlap fraction (4.83 g) boiling from 36-107 °C was taken and analyzed at 65.3 weight % butanol and 15.3 weight % EG. A final fraction (5.19 g) was distilled at 109-110°C/30 mm Hg and analyzed as 97.0 weight % EG and 1.2 weight % butanol. All of the fractions were colorless.

### Example 8: Depolymerization of Pet With N-butanol at a 1.25:1 Ratio

Pelletized bottle-flake waste PET (50.0 g; 0.276 mol) and potassium carbonate (1.01 g; 0.0072 mol; 0.028 equivalents) were slurried in n-butanol (62.5 g; 0.843 mol; 3.27 equivalents) in a 300-mL autoclave. The autoclave was pressurized with 500 psig nitrogen, then vented and sealed. Agitation was started and the autoclave was heated to 200 °C internal to generate about 120 psig autologous pressure. The vessel was further pressurized to 500 psig and stirred at 200 °C for four hours. The mixture was cooled to ambient temperature and poured out of the autoclave into a bottle (little remained in the autoclave). The non-viscous mixture was filtered to remove insolubles. HPLC analysis of the filtrate indicated 61% dibutyl terephthalate and the material analyzed at an acid number of 1.4. The theoretical content of the filtrate was 2.27 mmol total terephthalate per gram.

### Example 9: Depolymerization of Pet With 2-Ethylhexanol at a 10:1 Ratio

Pelletized bottle-flake waste PET (25.00 g; 130.1 mmol) and potassium carbonate (0.50 g; 3.6 mmol; 0.028 equivalents) were slurried in 2-ethylhexanol (250 g; 1920 mmol; 14.8 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate (235.45 g) was bottled. HPLC analysis of the filtrate indicated 91% bis(2-ethylhexyl) terephthalate and 8% 2-ethylhexyl hydroxyethyl terephthalate mixed ester. The theoretical content of the filtrate was 0.47 mmol total terephthalate per gram.

HPLC (150 x 4.6 mm Zorbax SB-C8 column, 90:10 (v:v) methanol: water (containing 0.1% trifluoroacetic acid) for 11 min, gradient to 100% methanol over 4 min, 250 nm detection): bis(2-ethylhexyl) terephthalate, t_{R} 9.95 min. ; mixed ester, t_{R} 2.64 min.

### Example 10: Transesterification of Pet 2-ethylhexanolysis Mixture (2-eh:Pet 10:1) With Methanol

PET 2-ethylhexanolysis mixture prepared as in Example 9 (0.47 mmol/g; 50 g; 0.024 mol) was slurried in 22.9 mL of methanol (0.565 mol; 24.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 50% aqueous sodium hydroxide (26 μL; 0.0005 mol; 0.02 equivalents) was added. The mixture was stirred at 250 rpm at 50 °C for 5 minutes and additional 0.02 equivalents of 50% sodium hydroxide was added. After 10 minutes an additional 0.04 equivalents of 50% sodium hydroxide was added and after 15 minutes 0.02 equivalents of 25% sodium methoxide in methanol was added. The mixture was stirred for a total of 45 minutes at 50 °C with no precipitation, and then was cooled to ambient temperature over 30 minutes, during which time a precipitate formed. An additional 0.05 equivalents of sodium methoxide in methanol was added and the mixture was stirred at ambient temperature for four hours. The resulting precipitate was filtered, washed with methanol, and air-dried to afford 1.78 g of DMT as a white powder which assayed at 96.2 weight % purity by HPLC, indicating 37% overall yield from PET.

### Example 11: Depolymerization of Pet With 2-Ethylhexanol at a 5:1 Ratio

Pelletized bottle-flake waste PET (25.00 g; 130.1 mmol) and potassium carbonate (0.50 g; 3.6 mmol; 0.028 equivalents) were slurried in 2-ethylhexanol (125 g; 960 mmol; 7.38 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate was bottled. HPLC analysis of the filtrate indicated 82% bis(2-ethylhexyl) terephthalate and 16% 2-ethylhexyl hydroxyethyl terephthalate mixed ester. The theoretical content of the filtrate was 0.86 mmol total terephthalate per gram.

### Example 12: Transesterification of PET 2-ethylhexanolysis mixture (2-EH:PET 5:1) With Methanol

PET 2-ethylhexanolysis mixture prepared as in Example 11 (0.86 mmol/g; 50.0 g; 0.043 mol) was slurried in 42.1 mL of methanol (1.039 mol; 24.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (198 μL; 0.0009 mol; 0.02 equivalents) was added. The mixture was stirred at 250 rpm at 50 °C for 5 minutes with no precipitate and an additional 0.03 equivalents of 25% sodium methoxide in methanol was added. Precipitation with an attendant exotherm was noted between 30 and 45 minutes. After heating for a total of 120 minutes at 50 °C the heating bath was then removed, and the mixture was allowed to cool to ambient temperature over 45 minutes and then stirred at ambient temperature for two hours. The resulting precipitate was filtered, washed with methanol, and air-dried to afford 5.68 g of DMT as a white powder which assayed at 97.8 weight % purity by HPLC, indicating 66% overall yield from PET. Analysis of the filtrate indicated that it contained 11.1% of the expected yield of DMT and small amounts (<0.5% each) of methyl hydroxyethyl terephthalate (MHET) and dimethyl isophthalate (DMI).

### Example 13: Depolymerization of Pet With 2-Ethylhexanol at a 2.5:1 Ratio

Pelletized bottle-flake waste PET (25.00 g; 130.1 mmol) and potassium carbonate (0.50 g; 3.6 mmol; 0.028 equivalents) were slurried in 2-ethylhexanol (62.5 g; 480 mmol; 3.69 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate was bottled. HPLC analysis indicated 69% bis(2-ethylhexyl) terephthalate and 25% 2-ethylhexyl hydroxyethyl terephthalate mixed ester.

### Example 14: Depolymerization of Pet With 2-Ethylhexanol at a 2:1 Ratio

Pelletized bottle-flake waste PET (25.00 g; 130.1 mmol) and potassium carbonate (0.50 g; 3.6 mmol; 0.028 equivalents) were slurried in 2-ethylhexanol (50.0 g; 384 mmol; 2.95 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate was bottled. The viscosity of the filtrate was analyzed and indicated a viscosity that decreased from 30.0 cP at 25°C to 10.8 cP at 60 °C. HPLC analysis of the filtrate indicated 64% bis(2-ethylhexyl) terephthalate and 28% 2-ethylhexyl hydroxyethyl terephthalate mixed ester. The theoretical content of the filtrate was 1.72 mmol total terephthalate per gram.

### Example 15: Transesterification of PET 2-Ethylhexanolysis Mixture With Methanol Using Sodium Hydroxide

PET 2-ethylhexanolysis mixture prepared as in Example 14 (1.72 mmol/g; 25 g; 0.043 mol) was slurried in 41.8 mL of methanol (1.032 mol; 24.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 50% aqueous sodium hydroxide (236 μL; 0.0043 mol; 0.10 equivalents) was added. The mixture was stirred at 200 rpm at 50 °C and stirred for 60 minutes during which time a precipitate was noted. The heating bath was then removed, and the mixture was allowed to cool to ambient temperature over 45 minutes and then stirred at ambient temperature for one hour. The resulting precipitate was filtered, washed with methanol, and air-dried to afford 5.07 g of DMT as a white powder which assayed at 99.9 weight % purity by HPLC, indicating 61% overall yield from PET. Analysis of the filtrate indicated that it contained 7.4% of the expected yield of DMT and small amounts (<0.5% each) of bis(hydroxyethyl) terephthalate (BHET), methyl hydroxyethyl terephthalate (MHET), and dimethyl isophthalate (DMI).

### Example 16: Depolymerization of Pet With 2-Ethylhexanol at a 1.5:1 Ratio

Pelletized bottle-flake waste PET (300.0 g; 1.56 mol) and potassium carbonate (6.04 g; 0.015 mol; 0.028 equivalents) were slurried in 2-ethylhexanol (450 g; 3.455 mol; 2.21 equivalents) in a 500-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate (229.1 g) was bottled. The residual solid was washed with methanol and dried to afford 2.5 g of insoluble residue. The viscosity of the filtrate was analyzed and indicated a viscosity that decreased from 43.8 cP at 25 °C to 14.7 cP at 60 °C. HPLC analysis of the filtrate indicated 47% bis(2-ethylhexyl) terephthalate and 28% 2-ethylhexyl hydroxyethyl terephthalate mixed ester, with the remainder being other esters and oligomers. The filtrate analyzed at an acid number of 1.36 and the theoretical content of the filtrate was 2.06 mmol total terephthalate per gram.

### Example 17: Transesterification of PET 2-Ethylhexanolysis mixture (2-EH:PET 1.5:1) With 24 Equivalents Methanol Using Sodium Methoxide Catalyst

PET 2-ethylhexanolysis mixture prepared as in Example 16 (2.06 mmol/g; 25.0 g; 0.052 mol) was slurried in 50.2 mL of methanol (1.239 mol; 24.0 equivalents) in a 300-mL 3-neck round-bottom flask with an overhead stirrer, a thermocouple, and an air condenser with a nitrogen inlet. The flask was placed into an oil bath set at 57°C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted within 20 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.53 g of DMT as a glistening white solid which assayed at 98.6 weight % purity by HPLC and GC, indicating 84% overall yield from PET. Analysis of the filtrate indicated that it contained 3.0% of the expected yield of DMT and small amounts (<0.5% each) of methyl hydroxyethyl terephthalate (MHET) and dimethyl isophthalate (DMI).

### Example 18: Transesterification of PET 2-Ethylhexanolysis mixture (2-EH:PET 1.5:1) With 21 Equivalents Methanol Using Sodium Methoxide Catalyst

PET 2-ethylhexanolysis mixture prepared as in Example 16 (2.06 mmol/g; 25.0 g; 0.052 mol) was slurried in 43.9 mL of methanol (1.084 mol; 21.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted within 20 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.88 g of DMT as a glistening white solid which assayed at 99.9 weight % purity by HPLC and GC, indicating 88% overall yield from PET. Analysis of the filtrate indicated that it contained 2.8% of the expected yield of DMT and small amounts (<0.5% each) of methyl hydroxyethyl terephthalate (MHET) and dimethyl isophthalate (DMI).

### Example 19: Transesterification of PET 2-Ethylhexanolysis mixture (2-EH:PET 1.5:1) With 18 Equivalents Methanol Using Sodium Methoxide Catalyst

PET 2-ethylhexanolysis mixture prepared as in Example 16 (2.06 mmol/g; 25.0 g; 0.052 mol) was slurried in 37.7 mL of methanol (0.929 mol; 18.0 equivalents) in a 300-mL 3-neck round-bottom flask with an overhead stirrer, a thermocouple, and an air condenser with a nitrogen inlet. The flask was placed into an oil bath set at 57°C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted at 10 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.97 g of DMT as a glistening white solid which assayed at 95.2 weight % purity by HPLC and GC, indicating 85% overall yield from PET. Analysis of the filtrate indicated that it contained 1.3% of the expected yield of DMT and small amounts (<0.5% each) of methyl hydroxyethyl terephthalate (MHET) and dimethyl isophthalate (DMI).

### Example 20: Transesterification of PET 2-ethylhexanolysis mixture (2-EH:PET 1.5:1) With 15 Equivalents Methanol Using Sodium Methoxide Catalyst

PET 2-ethylhexanolysis mixture prepared as in Example 16 (2.06 mmol/g; 25.0 g; 0.052 mol) was slurried in 31.3 mL of methanol (0.774 mol; 15.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted at nine minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.53 g of DMT as a glistening white solid which assayed at 98.0 weight % purity by HPLC and GC, indicating 83% overall yield from PET. Analysis of the filtrate indicated that it contained 1.6% of the expected yield of DMT and small amounts (<0.5% each) of methyl hydroxyethyl terephthalate (MHET) and dimethyl isophthalate (DMI).

### Example 21: Transesterification of PET 2-ethylhexanolysis mixture (2-EH:PET 1.5:1) With 12 Equivalents Methanol Using Sodium Methoxide Catalyst

PET 2-ethylhexanolysis mixture prepared as in Example 16 (2.06 mmol/g; 25.0 g; 0.052 mol) was slurried in 25.1 mL of methanol (0.620 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted within 20 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.37 g of DMT as a glistening white solid which assayed at 99.7 weight % purity by HPLC and GC, indicating 83% overall yield from PET. Analysis of the filtrate indicated that it contained 1.6% of the expected yield of DMT and small amounts (<0.5% each) of methyl hydroxyethyl terephthalate (MHET) and dimethyl isophthalate (DMI).

### Example 22: Transesterification of PET 2-Ethylhexanolysis Mixture (2-EH:PET 1.5:1) With 18 Equivalents Methanol Using Potassium Carbonate Catalyst

PET 2-ethylhexanolysis mixture prepared as in Example 16 (2.06 mmol/g; 25.0 g; 0.052 mol) was slurried in 37.7 mL of methanol (0.929 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) potassium carbonate (0.357 g; 0.0026 mol; 0.050 equivalents; 0.041 equivalents active catalyst based on acid number) was added. The mixture was stirred at 250 rpm at 50 °C and precipitation with an attendant exotherm was noted at seven minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.66 g of DMT as a glistening white solid which assayed at 98.5 weight % purity by HPLC and GC, indicating 85% overall yield from PET. Analysis of the filtrate indicated that it contained 2.5% of the expected yield of DMT and small amounts (<0.5% each) of methyl hydroxyethyl terephthalate (MHET) and dimethyl isophthalate (DMI).

### Example 23: Transesterification of PET 2-Ethylhexanolysis Mixture (2-EH:PET 1.5:1) With 18 Equivalents Methanol Using Sodium Methoxide Catalyst With Cooling

PET 2-ethylhexanolysis mixture prepared as in Example 16 (2.06 mmol/g; 25.0 g; 0.052 mol) was slurried in 37.7 mL of methanol (0.929 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (698 μL; 0.0031 mol; 0.059 equivalents; 0.050 equivalents active catalyst based on acid number) was added. The mixture was stirred at 250 rpm at 50 °C and precipitation with an attendant exotherm was noted at 10 minutes. After heating for a total of 60 minutes at 50 °C the heating bath was then removed and the mixture was allowed to cool to ambient temperature over 30 minutes and then stirred at ambient temperature for one hour. The mixture was then cooled in ice-water at 0-5 °C for one hour. The resulting precipitate was filtered, washed with methanol, and air-dried to afford 9.19 g of DMT as a glistening white solid which assayed at 96.0 weight % purity by HPLC and GC, indicating 88% overall yield from PET. Analysis of the filtrate indicated that it contained 1.2% of the expected yield of DMT and small amounts (<0.5% each) of methyl hydroxyethyl terephthalate (MHET) and dimethyl isophthalate (DMI).

### Example 24: Transesterification of PET 2-Ethylhexanolysis Mixture (2-EH:PET 1.5:1) With 18 Equivalents Methanol Using Potassium Carbonate Catalyst With Cooling

PET 2-ethylhexanolysis mixture prepared as in Example 16 (2.06 mmol/g; 25.0 g; 0.052 mol) was slurried in 37.7 mL of methanol (0.929 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) potassium carbonate (0.357 g; 0.0026 mol; 0.050 equivalents; 0.041 equivalents active catalyst based on acid number) was added. The mixture was stirred at 250 rpm at 50 °C and precipitation with an attendant exotherm was noted at 15 minutes. After heating for a total of 60 minutes at 50 °C the heating bath was then removed, and the mixture was allowed to cool to ambient temperature over 30 minutes and then stirred at ambient temperature for one hour. The mixture was then cooled in ice-water at 0-5 °C for one hour. The resulting precipitate was filtered, washed with methanol, and air-dried to afford 9.21 g of DMT as a glistening white solid which assayed at 93.6 weight % purity by HPLC and GC, indicating 86% overall yield from PET. Analysis of the filtrate indicated that it contained 1.2% of the expected yield of DMT and small amounts (<0.5% each) of methyl hydroxyethyl terephthalate (MHET) and dimethyl isophthalate (DMI).

### Example 25: Depolymerization of Carpet Pet With 2-ethylhexanol at a 1.5:1 Ratio

Pelletized carpet-derived waste PET (87.0% PET; 25 g; 113.2 mmol) and potassium carbonate (0.44 g; 3.2 mol; 0.028 equivalents) were slurried in 2-ethylhexanol (37.5 g; 287.9 mmol; 2.54 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate (48.39 g) was bottled. The residual solid was washed with methanol and dried to afford 5 g of insoluble residue. HPLC analysis of the filtrate indicated 58% bis(2-ethylhexyl) terephthalate and 33% 2-ethylhexyl hydroxyethyl terephthalate mixed ester, with the remainder being other esters and oligomers. The acid number of the filtrate was 2.48 and the theoretical content of the filtrate was 1.80 mmol total terephthalate per gram.

### Example 26: Transesterification of PET 2-Ethylhexanolysis Mixture From Carpet With 18 Equivalents Methanol Using Sodium Methoxide Catalyst

PET 2-ethylhexanolysis mixture prepared as in Example 25 (1.80 mmol/g; 43.0 g; 0.077 mol) was slurried in 56.4 mL of methanol (1.392 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (1.319 μL; 0.0058 mol; 0.075 equivalents; 0.050 equivalents active catalyst based on acid number) was added. The mixture was stirred at 250 rpm at 50 °C and precipitation with an attendant exotherm was noted at 30 minutes. After heating for a total of 60 minutes at 50 °C the heating bath was then removed, and the mixture was allowed to cool to ambient temperature over 30 minutes and then stirred at ambient temperature for two hours. The resulting precipitate was filtered, washed with methanol, and air-dried to afford 8.97 g of DMT as a glistening white solid which assayed at 99.9 weight % purity by HPLC and GC, indicating 81% overall yield from PET. Analysis of the filtrate indicated that it contained 9.4% of the expected yield of DMT and small amounts (<0.5% each) of methyl hydroxyethyl terephthalate (MHET) and dimethyl isophthalate (DMI).

### Example 27: Depolymerization of Auto Textile PET with 2-Ethylhexanol

Auto textile-derived waste PET (74.0% PET; 25.0 g; 96.3 mmol) and potassium carbonate (0.37 g; 2.7 mol; 0.028 equivalents) were slurried in 2-ethylhexanol (27.8 g; 213.1 mmol; 2.21 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate (29.38 g) was bottled. The residual solid was washed with methanol and dried to afford 13.87 g of insoluble residue. HPLC analysis of the filtrate indicated 49% bis(2-ethylhexyl) terephthalate and 24% 2-ethylhexyl hydroxyethyl terephthalate mixed ester, with the remainder being other esters and oligomers. The acid number of the filtrate (29.38 g) was measured at 1.40. The theoretical content of the filtrate was 2.06 mmol total terephthalate per gram.

### Example 28: Transesterification of PET 2-Ethylhexanolysis Mixture From Auto Textile With 18 Equivalents Methanol Using Sodium Methoxide Catalyst

PET 2-ethylhexanolysis mixture prepared as in Example 27 (2.06 mmol/g; 20.0 g; 0.0413 mol) was slurried in 30.1 mL of methanol (0.743 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (586 μL; 0.0026 mol; 0.062 equivalents; 0.05 equivalents active catalyst based on acid number) was added. The mixture was stirred at 250 rpm at 50 °C and precipitation with an attendant exotherm was noted at 10 minutes. After heating for a total of 45 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 6.32 g of DMT as a glistening white solid which assayed at 99.4 weight % purity by HPLC and GC, indicating 78% overall yield from PET. Analysis of the filtrate indicated that it contained 3.9% of the expected yield of DMT and small amounts (<0.5% each) of methyl hydroxyethyl terephthalate (MHET) and dimethyl isophthalate (DMI).

### Example 29: Depolymerization of Pet From Release Liners With 2-Ethylhexanol at a 1.5:1 Ratio

PET waste from siliconized release liners (98.0% PET; 150.0 g; 0.781 mol) and potassium carbonate (3.02 g; 0.022 mol; 0.028 equivalents) were slurried in 2-ethylhexanol (225.0 g; 1.728mol; 2.21 equivalents) in a 1-L 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered to afford 349.8 g of filtrate which was taken on directly. The residual solid was washed with methanol and dried to afford 10.53 g of insoluble residue. HPLC area % analysis of the filtrate indicated 58% bis(2-ethylhexyl) terephthalate, with the remainder being other esters and oligomers. The filtrate (59.7 g) was analyzed to have an acid number of 0.96. The theoretical content of the filtrate was 2.03 mmol total terephthalate per gram.

### Example 30: Transesterification of PET 2-Ethylhexanolysis Mixture From Release Liners With 18 Equivalents Methanol Using Sodium Methoxide Catalyst

PET 2-ethylhexanolysis mixture prepared as in Example 29 (349.8 g; 0.781 mol maximum) was slurried in 380 mL of methanol (9.37 mol; 12.0 equivalents) in a 1-L 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2°C) 25% sodium methoxide in methanol (10.71 mL; 0.047 mol; 0.06 equivalents) was added. The mixture was stirred at 250 rpm at 50 °C and precipitation with an attendant exotherm was noted at four minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 132.65 g of DMT as a glistening white solid which assayed at 91.0 weight % purity by HPLC, indicating 80% overall yield from PET. Analysis of the filtrate indicated that it contained 2.4% of the expected yield of DMT and small amounts (<0.5% each) of methyl hydroxyethyl terephthalate (MHET) and dimethyl isophthalate (DMI).

The filtrate from the DMT filtration was stripped of methanol at reduce pressure at 40 °C. The residue (259.33 g) was treated with 64.93 g of water. The layers were thoroughly mixed and allowed to settle. The layers were separated and analyzed. The top layer contained mostly 2-EH alcohol (83.45%) with a little bit of ethylene glycol (2.72%) and terephthalates. The bottom layer contained water, ethylene glycol (34.27%), and a small amount of 2-ethylhexanol (0.67%). The bottom layer was sequentially distilled, first at atmospheric pressure to remove water and the water/2-ethylhexanol azeotrope, then under vacuum to distill ethylene glycol (17.9 g). The distilled ethylene glycol was assayed at 98.0% purity and had an APHA color of 4.5. Analysis for silicon indicated that the crystallized DMT had 104 ppm Si and the distilled EG had 646 ppm Si.

### Example 31: Depolymerization of Pet With 2-Ethylhexanol at a 1.25:1 Ratio

Pelletized bottle-flake waste PET (25.00 g; 130.1 mmol) and potassium carbonate (0.50 g; 3.6 mmol; 0.028 equivalents) were slurried in 2-ethylhexanol (31.3 g; 240 mmol; 1.84 equivalents) in a 300-mL 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate was bottled (46.06 g). HPLC analysis of the filtrate indicated 41% bis(2-ethylhexyl) terephthalate and 28% 2-ethylhexyl hydroxyethyl terephthalate mixed ester, with the remainder being other esters and oligomers.

### Example 32: Depolymerization of PET with 2-Ethylhexanol at a 1.5:1 Ratio With Butyltin Tris(2-Ethylhexanoate) Catalyst

Pelletized bottle-flake waste PET (25.00 g; 130.1 mmol) and butyltin tris(2-ethylhexanoate) (FASCAT® 4102 (PMC), 1.57 g; 2.6 mmol; 0.02 equivalents) were slurried in 2-ethylhexanol (37.5 g; 288 mmol; 2.21 equivalents) in a 300-mL 3-neck round-bottom flask with an overhead stirrer, a Dean-Stark trap with a condenser, and a nitrogen inlet. The flask was heated to 185 °C internal and held for 2h after full dissolution of the PET (required 3.5 h to dissolve). The mixture was cooled to 60 °C and filtered, and the filtrate was bottled (66.11 g). HPLC analysis of the filtrate indicated 53% bis(2-ethylhexyl) terephthalate and 30% 2-ethylhexyl hydroxyethyl terephthalate mixed ester, with the remainder being other esters and oligomers. The theoretical content of the filtrate was 2.03 mmol total terephthalate per gram.

### Example 33: Transesterification of PET 2-Ethylhexanolysis mixture (2-EH:PET 1.5:1) Prepared With Butyltin Tris(2-Ethylhexanoate)

PET 2-ethylhexanolysis mixture prepared as in Example 32 (2.03 mmol/g; 25.0 g; 0.051 mol) with an acid number of 1.95 was slurried in 37.0 mL of methanol (0.914 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (778 μL; 0.0034 mol; 0.067 equivalents; 0.050 equivalents active catalyst based on acid number) was added. The mixture was stirred at 250 rpm at 50 °C and precipitation with an attendant exotherm was noted at 12 minutes. After heating for a total of 60 minutes at 50 °C the heating bath was then removed, and the mixture was allowed to cool to ambient temperature over 45 minutes and then stirred at ambient temperature for one hour. The resulting precipitate was filtered, washed with methanol, and air-dried to afford 7.95 g of DMT as a glistening white solid which assayed at 99.4 weight % purity by HPLC and GC, indicating 81% overall yield from PET. Analysis of the filtrate indicated that it contained 5.9% of the expected yield of DMT and small amounts (<0.5% each) of bis(hydroxyethyl) terephthalate (BHET), methyl hydroxyethyl terephthalate (MHET) and dimethyl isophthalate (DMI).

### Example 34: Depolymerization of Pet With 2-Ethylhexanol at a 1.5:1 Ratio With Titanium Tetra(Isopropoxide) Catalyst

Pelletized bottle-flake waste PET (25.00 g; 130.1 mmol) and titanium tetra(isopropoxide) (0.38 g; 1.3 mmol; 0.01 equivalents) were slurried in 2-ethylhexanol (37.5 g; 288 mmol; 2.21 equivalents) in a 300-mL 3-neck round-bottom flask with an overhead stirrer, a Dean-Stark trap with a condenser, and a nitrogen inlet. The flask was heated to 185 °C internal and held for 2 h after full dissolution of the PET. The mixture was cooled to 60 °C and filtered, and the filtrate was bottled (58.6 g). HPLC analysis of the filtrate indicated 51% bis(2-ethylhexyl) terephthalate and 29% 2-ethylhexyl hydroxyethyl terephthalate mixed ester, with the remainder being other esters and oligomers. The theoretical content of the filtrate was 2.07 mmol total terephthalate per gram.

### Example 35: Transesterification of Pet 2-Ethylhexanolysis Mixture (2-eh:Pet 1.5:1) Prepared With Ti(OiPr)₄

PET 2-ethylhexanolysis mixture prepared as in Example 34 (2.07 mmol/g; 25.0 g; 0.052 mol) with an acid number of 1.61 was slurried in 37.8 mL of methanol (0.932 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (756 μL; 0.0033 mol; 0.064 equivalents; 0.050 equivalents active catalyst based on acid number) was added. The mixture was stirred at 250 rpm at 50 °C and precipitation with an attendant exotherm was noted within 20 minutes. After heating for a total of 60 minutes at 50 °C the heating bath was then removed, and the mixture was allowed to cool to ambient temperature over 45 minutes and then stirred at ambient temperature for one hour. The resulting precipitate was filtered, washed with methanol, and air-dried to afford 8.37 g of DMT as a glistening white solid which assayed at 99.9 weight % purity by HPLC and GC, indicating 83% overall yield from PET. Analysis of the filtrate indicated that it contained 4.3% of the expected yield of DMT and small amounts (<0.5% each) of methyl hydroxyethyl terephthalate (MHET) and dimethyl isophthalate (DMI).

### Example 36: Depolymerization of Pet With 2-Ethylhexanol at a 1.5:1 Ratio With Sodium Bicarbonate Catalyst

Pelletized bottle-flake waste PET (25.00 g; 130.1 mmol) and sodium bicarbonate (0.31 g; 3.6 mmol; 0.028 equivalents) were slurried in 2-ethylhexanol (37.5 g; 288 mmol; 2.21 equivalents) in a 300-mL 3-neck round-bottom flask with an overhead stirrer, a Dean-Stark trap with a condenser, and a nitrogen inlet. The flask was heated to 185 °C internal and stirred for six total hours (required 2.5 h to dissolve the PET). The mixture was cooled to 60 °C and filtered to remove an insoluble gel-like material (3.27 g), and the filtrate was bottled (50.87 g). HPLC analysis of the filtrate indicated 56% bis(2-ethylhexyl) terephthalate and 31% 2-ethylhexyl hydroxyethyl terephthalate mixed ester, with the remainder being other esters and oligomers. The theoretical content of the filtrate was 2.07 mmol total terephthalate per gram and the filtrate had an acid number of 1.08.

### Example 37: Transesterification of Pet 2-Ethylhexanolysis Mixture (2-eh:Pet 1.5:1) Prepared Using Sodium Bicarbonate

PET 2-ethylhexanolysis mixture prepared as in Example 36 (2.07 mmol/g; 25.0 g; 0.052 mol) was slurried in 37.8 mL of methanol (0.932 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (702 μL; 0.0031 mol; 0.059 equivalents; 0.050 equivalents active catalyst based on acid number) was added. The mixture was stirred at 250 rpm at 50 °C and precipitation with an attendant exotherm was noted at seven minutes. After heating for a total of 60 minutes at 50 °C the heating bath was then removed, and the mixture was allowed to cool to ambient temperature over 30 minutes and then stirred at ambient temperature for 90 minutes. The resulting precipitate was filtered, washed with methanol, and air-dried to afford 8.69 g of DMT as a glistening white solid which assayed at >99.9 weight % purity by HPLC and GC, indicating 86% overall yield from PET. Analysis of the filtrate indicated that it contained 2.9% of the expected yield of DMT and small amounts (<0.5% each) of bis(hydroxyethyl) terephthalate (BHET), methyl hydroxyethyl terephthalate (MHET) and dimethyl isophthalate (DMI).

### Example 38: Depolymerization of Pet With 2-Ethylhexanol at a 1.5:1 Ratio With Potassium Bicarbonate Catalyst

Pelletized bottle-flake waste PET (25.00 g; 130.1 mmol) and potassium bicarbonate (0.36 g; 3.6 mmol; 0.028 equivalents) were slurried in 2-ethylhexanol (37.5 g; 288 mmol; 2.21 equivalents) in a 300-mL 3-neck round-bottom flask with an overhead stirrer, a Dean-Stark trap with a condenser, and a nitrogen inlet. The flask was heated to 185 °C internal and stirred for six total hours (required 2 h to dissolve the PET). The mixture was cooled to 60 °C and filtered (insoluble material <1 g), and the filtrate was bottled (66.11 g). HPLC analysis of the filtrate indicated 55% bis(2-ethylhexyl) terephthalate and 33% 2-ethylhexyl hydroxyethyl terephthalate mixed ester, with the remainder being other esters and oligomers. The theoretical content of the filtrate was 2.07 mmol total terephthalate per gram and the filtrate had an acid number of 1.37.

### Example 39: Transesterification of PET 2-Ethylhexanolysis Mixture (2-EH:PET 1.5:1) Prepared With Potassium Bicarbonate

PET 2-ethylhexanolysis mixture prepared as in Example 38 (2.07 mmol/g; 25.0 g; 0.052 mol) was slurried in 37.8 mL of methanol (0.932 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (731 μL; 0.0032 mol; 0.062 equivalents; 0.050 equivalents active catalyst based on acid number) was added. The mixture was stirred at 250 rpm at 50 °C and precipitation with an attendant exotherm was noted at 7.5 minutes. After heating for a total of 60 minutes at 50 °C the heating bath was then removed, and the mixture was allowed to cool to ambient temperature over 30 minutes and then stirred at ambient temperature for 2 hours. The resulting precipitate was filtered, washed with methanol, and air-dried to afford 7.74 g of DMT as a glistening white solid which assayed at >99.9 weight % purity by HPLC and GC, indicating 77% overall yield from PET. Analysis of the filtrate indicated that it contained 2.3% of the expected yield of DMT and small amounts (<0.5% each) of bis(hydroxyethyl) terephthalate (BHET), methyl hydroxyethyl terephthalate (MHET) and dimethyl isophthalate (DMI).

### Example 40: Depolymerization of PET With 1-hexanol at a 2:1 Ratio

Pelletized bottle-flake waste PET (25.00 g; 130.1 mmol) and potassium carbonate (0.50 g; 3.6 mmol; 0.028 equivalents) were slurried in 1-hexanol (50 g; 489 mmol; 3.76 equivalents) in a 300-mL 3-neck round-bottom flask with an overhead stirrer, a Dean-Stark trap with a condenser, and a nitrogen inlet. The flask was heated to 157 °C internal and held for 1 h after full dissolution of the PET. The mixture was cooled to 60 °C and filtered, and the precipitate was washed and dried to afford 2.90 g. The filtrate was bottled (66.18 g). HPLC analysis of the filtrate indicated 65% dihexyl terephthalate with the remainder being other esters and oligomers. The theoretical content of the filtrate was 1.72 mmol total terephthalate per gram.

### Example 41: Transesterification of Pet Hexanolysis Mixture (Hexanol: Pet 2:1) With Methanol

PET hexanolysis mixture prepared as in Example 40 (1.72 mmol/g; 25.0 g; 0.043 mol) was slurried in 41.9 mL of methanol (1.034 mol; 24.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2°C) 25% sodium methoxide in methanol (492 μL; 0.0022 mol; 0.05 equivalents) was added. The mixture was stirred at 250 rpm at 50 °C and precipitation with an attendant exotherm was noted at seven minutes. After heating for a total of 60 minutes at 50 °C the heating bath was then removed, and the mixture was allowed to cool to ambient temperature over 30 minutes and then stirred at ambient temperature for two hours. The resulting precipitate was filtered, washed with methanol, and air-dried to afford 5.90 g of DMT as a white solid which assayed at 98.3 weight % purity by GC, indicating 69% overall yield from PET. Analysis of the filtrate indicated that it contained 15.0% of the expected yield of DMT and small amounts (<0.5% each) of methyl hydroxyethyl terephthalate (MHET) and dimethyl isophthalate (DMI).

### Example 42: Depolymerization of Pet With 1-Octanol at a 1.5:1 Ratio

Pelletized bottle-flake waste PET (25.00 g; 130.1 mmol) and potassium carbonate (0.50 g; 3.6 mmol; 0.028 equivalents) were slurried in 1-octanol (37.5 g; 288 mmol; 2.21 equivalents) in a 300-mL 3-neck round-bottom flask with an overhead stirrer, a Dean-Stark trap with a condenser, and a nitrogen inlet. The flask was heated to 190 °C internal and stirred for 1.5 h to afford full dissolution of the PET and held under conditions for a further one hour. The mixture was cooled to 60 °C and filtered, and the precipitate was washed and dried to afford 1.52 g. The filtrate was bottled (52.74 g). HPLC analysis of the filtrate indicated 61% dioctyl terephthalate with the remainder being other mixed esters and oligomers. The theoretical content of the filtrate was 2.06 mmol total terephthalate per gram.

### Example 43: Transesterification of Pet Octanolysis Mixture (octanol:Pet 1.5:1) With Methanol

PET octanolysis mixture prepared as in Example 42 (2.06 mmol/g; 25.0 g; 0.052 mol) was slurried in 37.7 mL of methanol (0.929 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2°C) 25% sodium methoxide in methanol (708 L; 0.0031 mol; 0.06 equivalents) was added. The mixture was stirred at 250 rpm at 50 °C and precipitation with an attendant exotherm was noted at three minutes. After heating for a total of 60 minutes at 50 °C during which time the mixture became a very thick slurry the heating bath was then removed, and the mixture was allowed to cool to ambient temperature over 30 minutes and then stirred at ambient temperature for one hour. The resulting precipitate was filtered, washed with methanol, and air-dried to afford 5.90 g of DMT as a white powder which assayed at 99.8 weight % purity by GC, indicating 92% overall yield from PET. Analysis of the filtrate indicated that it contained 2.5% of the expected yield of DMT and small amounts (<0.5% each) of methyl hydroxyethyl terephthalate (MHET) and dimethyl isophthalate (DMI).

### Example 44: Depolymerization of Pet With 1-decanol at a 2:1 Ratio

Pelletized bottle-flake waste PET (25.00 g; 130.1 mmol) and potassium carbonate (0.50 g; 3.6 mmol; 0.028 equivalents) were slurried in 1-decanol (50.0 g; 315.9 mmol; 2.43 equivalents) in a 300-mL 3-neck round-bottom flask with an overhead stirrer, a Dean-Stark trap with a condenser, and a nitrogen inlet. The flask was heated to 190 °C internal and stirred for 3 h to afford nearly complete dissolution of the PET. The mixture was cooled to 65 °C and filtered through a warm fritted funnel. The filtrate was bottled and solidified upon cooling to ambient temperature (70.74 g). The theoretical content of the filtrate was 1.72 mmol total terephthalate per gram.

### Example 45: Transesterification of Pet Decanolysis Mixture (decanol:Pet 2:1) With Methanol

PET decanolysis mixture prepared as in Example 44 (1.72 mmol/g; 30.0 g; 0.052 mol) was slurried in 37.7 mL of methanol (0.930 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (709 μL; 0.0031 mol; 0.06 equivalents) was added. The mixture was stirred at 250 rpm at 50 °C and precipitation with an attendant exotherm was noted within four minutes. After heating for a total of 60 minutes at 50 °C during which time the mixture became a very thick slurry the heating bath was then removed, and the mixture was allowed to cool to ambient temperature over 45 minutes and then stirred at ambient temperature for one hour. The resulting precipitate was filtered, washed with methanol, and air-dried to afford 8.62 g of DMT as a white powder which assayed at 99.3 weight % purity by LC, indicating 85% overall yield from PET. Analysis of the filtrate indicated that it contained 4.6% of the expected yield of DMT and small amounts (<0.5% each) of bis(hydroxyethyl) terephthalate (BHET, methyl hydroxyethyl terephthalate (MHET) and dimethyl isophthalate (DMI).

### Example 46: Depolymerization of PET With a Mixture of Octanol and Decanol at an Alcohol: PET Ratio of 2:1

Pelletized bottle-flake waste PET (25.00 g; 130.1 mmol) and potassium carbonate (0.50 g; 3.6 mmol; 0.028 equivalents) were slurried in a 55:45 mixture of 1-octanol (27.5 g; 211 mmol; 1.62 equivalents) and 1-decanol (22.5 g; 142.2 mmol; 1.09 equivalents) respectively in a 300-mL 3-neck round-bottom flask with an overhead stirrer, a Dean-Stark trap with a condenser, and a nitrogen inlet. The flask was heated to reflux (178-182 °C) and stirred for 3 h at which point no obvious PET remained. The mixture was cooled to 65 °C and the mixture was filtered through a pre-heated (50 °C) fritted funnel with filter paper. There was very little precipitate, and the filtrate was bottled (71.44 g). The theoretical content of the filtrate was 1.72 mmol total terephthalate per gram.

### Example 47: Transesterification of Pet Octanolysis/decanolysis Mixture (alcohol:PET 2:1) With Methanol

PET octanolysis/decanolysis mixture prepared as in Example 46 (1.72 mmol/g; 30.0 g; 0.052 mol) was slurried in 37.7 mL of methanol (930 mmol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction solution equilibrated at 50 °C (+/−2 °C) 25% sodium methoxide in methanol (709 μL; 0.0031 mol; 0.06 equivalents) was added. The mixture was stirred at 250 rpm at 50 °C and at 3.5 minutes precipitation with an attendant exotherm was noted. After heating for a total of 60 minutes at 50 °C during which time the mixture became a thick slurry, the heating bath was then removed, and the mixture was allowed to cool to ambient temperature over 45 minutes and then stirred at ambient temperature for one hour. The resulting precipitate was filtered, washed with methanol, and air-dried to afford 8.44 g of DMT as a white powder which assayed at 99.8 weight % purity by HPLC and GC, indicating 84% overall yield from PET. Analysis of the filtrate indicated that it contained 4.0% of the expected yield of DMT and small amounts (<0.5% each) of bis(hydroxyethyl) terephthalate (BHET), methyl hydroxyethyl terephthalate (MHET) and dimethyl isophthalate (DMI).

### Example 48: Depolymerization of PET with dodecanol at a 1.5:1 Ratio

Pelletized bottle-flake waste PET (25.00 g; 130.1 mmol) and potassium carbonate (0.50 g; 3.6 mmol; 0.028 equivalents) were slurried in 1-dodecanol (37.5 g; 201 mmol; 1.55 equivalents) in a 300-mL 3-neck round-bottom flask with an overhead stirrer, a Dean-Stark trap with a condenser, and a nitrogen inlet. The flask was heated to 190 °C internal and stirred for 6 h at which point a small amount of PET was noted. The mixture was cooled to 70 °C and the liquid was decanted from the residue to afford 52.32 g which solidified at ambient temperature. HPLC analysis of the decanted material indicated 50% dilauryl terephthalate with the remainder being other mixed esters and oligomers. The theoretical content of the filtrate was 2.06 mmol total terephthalate per gram.

### Example 49: Transesterification of Pet Dodecanolysis Mixture (dodecanol:PET 1.5:1) With Methanol

PET dodecanolysis mixture prepared as in Example 48 (2.06 mmol/g; 25.0 g; 0.052 mol) was slurried in 43.9 mL of methanol (1.084 mol; 21.0 equivalents) in a 300-mL 3-neck round-bottom flask with an overhead stirrer, a thermocouple, and an air condenser with a nitrogen inlet. The flask was placed into an oil bath set at 57 °C, and once the reaction slurry equilibrated at 50 °C (+/−2 °C) 25% sodium methoxide in methanol (708 μL; 0.0031 mol; 0.06 equiv) was added. The mixture was stirred at 250 rpm at 50 °C for 60 min. This mixture remained a slurry during the full hold time, although the consistency of the slurry did change. After heating for a total of 60 minutes at 50 °C the heating bath was then removed, and the mixture was allowed to cool to ambient temperature over 45 minutes and then stirred at ambient temperature overnight. The resulting precipitate was filtered, washed with methanol, and air-dried to afford 9.44 g of DMT as a glistening white solid which assayed at 99.4 weight % purity by HPLC and GC, indicating 94% overall yield from PET. Analysis of the filtrate indicated that it contained 0.3% of the expected yield of DMT and small amounts (<0.5% each) of bis(hydroxyethyl) terephthalate (BHET), methyl hydroxyethyl terephthalate (MHET) and dimethyl isophthalate (DMI).

### Example 50: Depolymerization of Pet With Tetradecanol at a 2:1 Ratio

Pelletized bottle-flake waste PET (25.00 g; 130.1 mmol) and potassium carbonate (0.50 g; 3.6 mmol; 0.028 equivalents) were combined with 1-tetradecanol (50.0 g; 201 mmol; 1.55 equivalents) in a 300-mL 3-neck round-bottom flask with an overhead stirrer, a Dean-Stark trap with a condenser, and a nitrogen inlet. The flask was heated to 190 °C internal and stirred for 2h at which point a small amount of PET was noted and the reaction had appeared to stall. The mixture was cooled to 75 °C and the stirring was stopped. The liquid was decanted from the residue, and the liquid set up immediately as a waxy solid (64.98 g). The theoretical content of the filtrate was 1.72 mmol total terephthalate per gram.

### Example 51: Transesterification of PET Tetradecanolysis Mixture (Tetradecanol:PET 2:1) With Methanol

PET tetradecanolysis mixture prepared as in Example 50 (1.72 mmol/g; 30.0 g; 0.052 mol) was slurried in 37.7 mL of methanol (930 mol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction slurry equilibrated at 50 °C (+/−2 °C) 25% sodium methoxide in methanol (709 μL; 0.0031 mol; 0.06 equivalents) was added. The mixture was stirred at 250 rpm at 50 °C for 60 minutes. This mixture remained a slurry during the full hold time, although the consistency of the slurry did change as it became much thicker. After heating for a total of 60 minutes at 50 °C the heating bath was then removed, and the mixture was allowed to cool to ambient temperature over 45 minutes and then stirred at ambient temperature for 1 h. The resulting precipitate was filtered, washed with methanol, and air-dried to afford 12.44 g of DMT as a white solid which assayed at 73.8 weight % purity by HPLC and GC, indicating 91% overall yield from PET. Analysis of the filtrate indicated that it contained small amounts (<0.5% each) of bis(hydroxyethyl) terephthalate (BHET), methyl hydroxyethyl terephthalate (MHET) and dimethyl isophthalate (DMI).

### Example 52: Depolymerization of Pet With a Mixture of Dodecanol and Tetradecanol at a 2:1 Ratio

Pelletized bottle-flake waste PET (25.00 g; 130.1 mmol) and potassium carbonate (0.50 g; 3.6 mmol; 0.028 equivalents) were slurried in a 70:30 mixture of dodecanol (35.0 g; 188 mmol; 1.44 equivalents) and tetradecanol (15.0 g; 70.0 mmol; 0.54 equivalents) in a 300-mL 3-neck round-bottom flask with an overhead stirrer, a Dean-Stark trap with a condenser, and a nitrogen inlet. The flask was heated to 190 °C internal and stirred for 4 h at which point a small amount of PET was noted. The mixture was cooled to 75 °C and the stirring was stopped. The liquid was decanted from the residue to afford 70.69 g, and the liquid set up into a solid at about 40 °C. The theoretical content of the filtrate was 1.72 mmol total terephthalate per gram.

### Example 53: Transesterification of PET Dodecanolysis/Tetradecanolysis Mixture (Alcohol:PET 2:1) With Methanol

PET dodecanolysis/tetradecanolysis mixture prepared as in Example 52 (1.72 mmol/g; 30.0 g; 0.052 mol) was slurried in 37.7 mL of methanol (930 mmol; 18.0 equivalents) in a 300-mL 3-neck round-bottom flask with an overhead stirrer, a thermocouple, and an air condenser with a nitrogen inlet. The flask was placed into an oil bath set at 57 °C, and once the reaction slurry equilibrated at 50 °C (+/−2 °C) 25% sodium methoxide in methanol (709 μL; 0.0031 mol; 0.06 equivalents) was added. The mixture was stirred at 250 rpm at 50 °C. After about two minutes the mixture turned into a clear solution, and at 4.5 minutes precipitation with an attendant exotherm was noted. After heating for a total of 60 minutes at 50 °C during which time the mixture became a very thick slurry the heating bath was then removed, and the mixture was allowed to cool to ambient temperature over 45 minutes and then stirred at ambient temperature for two hours. The resulting precipitate was filtered, washed with methanol, and air-dried to afford 8.76 g of DMT as a white powder which assayed at 98.2 weight % purity by HPLC and GC, indicating 86% overall yield from PET. Analysis of the filtrate indicated that it contained 4.7% of the expected yield of DMT and small amounts (<0.5% each) of bis(hydroxyethyl) terephthalate (BHET), methyl hydroxyethyl terephthalate (MHET) and dimethyl isophthalate (DMI).

### Example 54: Depolymerization of PET With 2-ethylhexanol Using Sodium Acetate Catalyst

Pelletized bottle-flake waste PET (25.00 g; 0.1276 mol) and sodium acetate (0.52 g; 0.0064 mol; 0.05 equivalents) were slurried in 2-ethylhexanol (37.5 g; 0.2879 mol; 2.26 equivalents) in a 250-mL 3-neck round-bottom flask with an overhead stirrer, a Dean-Stark trap with a condenser, and a nitrogen inlet. The flask was heated to 185 °C internal and held for 10 h (1 h after full dissolution of the PET). The filtrate analyzed at an acid by-product content of 2.36% by HPLC area% and the theoretical content of the filtrate was 2.02 mmol total terephthalate per gram.

### Example 55: Transesterification of PET 2-Ethylhexanolysis Mixture (Sodium Acetate Catalyst) With 12 Equivalents Methanol Using Sodium Methoxide Catalyst

PET 2-ethylhexanolysis mixture prepared as in Example 54 (2.02 mmol/g; 15.00 g; 0.0304 mol) was slurried in 14.8 mL of methanol (0.365 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (257 μL; 0.0011 mol; 0.037 equivalents) was added. The mixture was stirred at 250 rpm at 50 °C and precipitation with an attendant exotherm was noted at 9.5 minutes. After heating for a total of 60 minutes at 50 °C the heating bath was then removed, and the mixture was allowed to cool to ambient temperature over 20 minutes and then stirred at ambient temperature for one hour. The resulting precipitate was filtered, washed with methanol, and air-dried to afford 4.72 g of DMT as a glistening white solid which assayed at 98.7 weight % purity by HPLC and GC, indicating 79% overall yield from PET. Analysis of the filtrate indicated that it contained 13% of the expected yield of DMT and small amounts (<0.5% each) of methyl hydroxyethyl terephthalate (MHET) and dimethyl isophthalate (DMI).

### Example 56: Depolymerization of Pet With 2-ethylhexanol Using 2.5 mol % Potassium Acetate Catalyst

Pelletized bottle-flake waste PET (98.1% PET; 20.39 g; 0.1041 mol) and potassium acetate (0.255 g; 0.0026 mol; 0.025 equivalents) were slurried in 2-ethylhexanol (30.6 g; 0.2349 mol; 2.26 equivalents) in a 250-mL 3-neck round-bottom flask with an overhead stirrer, a Dean-Stark trap with a condenser, and a nitrogen inlet. The flask was heated to 185 °C internal and held for 3 h (1 h after full dissolution of the PET). The filtrate analyzed at an acid by-product content of 2.62% by HPLC area% and the theoretical content of the filtrate was 2.03 mmol total terephthalate per gram.

### Example 57: Transesterification of PET 2-Ethylhexanolysis Mixture (2.5 mol % Potassium Acetate Catalyst) With 12 Equiv Methanol Using Sodium Methoxide Catalyst

PET 2-ethylhexanolysis mixture prepared as in Example 56 (2.03 mmol/g; 25.00 g; 0.0508 mol) was slurried in 24.7 mL of methanol (0.609 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (580 μL; 0.0025 mol; 0.05 equivalents) was added. The mixture was stirred at 250 rpm at 50 °C and precipitation with an attendant exotherm was noted at 5 minutes. After heating for a total of 60 minutes at 50 °C the heating bath was then removed, and the mixture was allowed to cool to ambient temperature over 30 minutes and then stirred at ambient temperature for two hours. The resulting precipitate was filtered, washed with methanol, and air-dried to afford 9.18 g of DMT as a glistening white solid which assayed at 99.4 weight % purity by HPLC and GC, indicating 93% overall yield from PET. Analysis of the filtrate indicated that it contained 1.2% of the expected yield of DMT and small amounts (<0.5% each) of methyl hydroxyethyl terephthalate (MHET) and dimethyl isophthalate (DMI).

### Example 58: Depolymerization of Pet With 2-ethylhexanol Using 2.0 mol % Potassium Acetate Catalyst

Pelletized bottle-flake waste PET (98.1% PET; 20.39 g; 0.1041 mol) and potassium acetate (0.204 g; 0.0021 mol; 0.02 equivalents) were slurried in 2-ethylhexanol (30.6 g; 0.2349 mol; 2.26 equivalents) in a 250-mL 3-neck round-bottom flask with an overhead stirrer, a Dean-Stark trap with a condenser, and a nitrogen inlet. The flask was heated to 185 °C internal and held for 3 h (1 h after full dissolution of the PET). The filtrate analyzed at an acid by-product content of 2.40% by HPLC area % and the theoretical content of the filtrate was 2.03 mmol total terephthalate per gram.

### Example 59: Transesterification of PET 2-Ethylhexanolysis Mixture (2.0 mol % Potassium Acetate Catalyst) With 12 Equiv Methanol Using Sodium Methoxide Catalyst

PET 2-ethylhexanolysis mixture prepared as in Example 58 (2.03 mmol/g; 25.00 g; 0.0508 mol) was slurried in 24.7 mL of methanol (0.609 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (580 μL; 0.0025 mol; 0.05 equivalents) was added. The mixture was stirred at 250 rpm at 50 °C and precipitation with an attendant exotherm was noted at 5 minutes. After heating for a total of 60 minutes at 50 °C the heating bath was then removed, and the mixture was allowed to cool to ambient temperature over 30 minutes and then stirred at ambient temperature for 45 minutes. The resulting precipitate was filtered, washed with methanol, and air-dried to afford 9.02 g of DMT as a glistening white solid which assayed at 99.8 weight % purity by HPLC and GC, indicating 91% overall yield from PET. Analysis of the filtrate indicated that it contained 3.6% of the expected yield of DMT and small amounts (<0.5% each) of methyl hydroxyethyl terephthalate (MHET) and dimethyl isophthalate (DMI).

### Example 60: Depolymerization of Pet With 2-Ethylhexanol Using 1.5 mol % Potassium Acetate Catalyst

Pelletized bottle-flake waste PET (98.1% PET; 20.39 g; 0.1041 mol) and potassium acetate (0.153 g; 0.0016 mol; 0.015 equivalents) were slurried in 2-ethylhexanol (30.6 g; 0.2349 mol; 2.26 equivalents) in a 250-mL 3-neck round-bottom flask with an overhead stirrer, a Dean-Stark trap with a condenser, and a nitrogen inlet. The flask was heated to 185 °C internal and held for 4 h (1 h after full dissolution of the PET). The filtrate analyzed at an acid by-product content of 2.08% by HPLC area% and the theoretical content of the filtrate was 2.04 mmol total terephthalate per gram.

### Example 61: Transesterification of PET 2-Ethylhexanolysis Mixture (1.5 mol % Potassium Acetate Catalyst) With 12 Equivalents Methanol Using Sodium Methoxide Catalyst

PET 2-ethylhexanolysis mixture prepared as in Example 60 (2.04 mmol/g; 25.00 g; 0.0510 mol) was slurried in 24.8 mL of methanol (0.612 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50°C (+/−2 °C) 25% sodium methoxide in methanol (583 μL; 0.0026 mol; 0.05 equivalents) was added. The mixture was stirred at 250 rpm at 50 °C and precipitation with an attendant exotherm was noted at 4.5 minutes. After heating for a total of 60 minutes at 50 °C the heating bath was then removed, and the mixture was allowed to cool to ambient temperature over 30 minutes and then stirred at ambient temperature for 45 minutes. The resulting precipitate was filtered, washed with methanol, and air-dried to afford 9.24 g of DMT as a glistening white solid which assayed at 99.9 weight % purity by HPLC and GC, indicating 93% overall yield from PET. Analysis of the filtrate indicated that it contained 2.9% of the expected yield of DMT and small amounts (<0.5% each) of methyl hydroxyethyl terephthalate (MHET) and dimethyl isophthalate (DMI).

### Example 62: Depolymerization of Pet With 2-Ethylhexanol Using 1.25 mol % Potassium Acetate Catalyst

Pelletized bottle-flake waste PET (98.1% PET; 20.39 g; 0.1041 mol) and potassium acetate (0.128 g; 0.0016 mol; 0.0125 equivalents) were slurried in 2-ethylhexanol (30.6 g; 0.2349 mol; 2.26 equivalents) in a 250-mL 3-neck round-bottom flask with an overhead stirrer, a Dean-Stark trap with a condenser, and a nitrogen inlet. The flask was heated to 185 °C internal and held for 5 h (1 h after full dissolution of the PET). The filtrate analyzed at an acid by-product content of 1.58% by HPLC area % and the theoretical content of the filtrate was 2.04 mmol total terephthalate per gram.

### Example 63: Transesterification of PET 2-Ethylhexanolysis Mixture (1.25 mol % Potassium Acetate Catalyst) With 12 Equiv Methanol Using Sodium Methoxide Catalyst

PET 2-ethylhexanolysis mixture prepared as in Example 62 (2.04 mmol/g; 25.00 g; 0.0510 mol) was slurried in 24.8 mL of methanol (0.612 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (583 μL; 0.0026 mol; 0.05 equivalents) was added. The mixture was stirred at 250 rpm at 50 °C and precipitation with an attendant exotherm was noted at 4.5 minutes. After heating for a total of 60 minutes at 50 °C the heating bath was then removed, and the mixture was allowed to cool to ambient temperature over 30 minutes and then stirred at ambient temperature for 90 minutes. The resulting precipitate was filtered, washed with methanol, and air-dried to afford 9.61 g of DMT as a glistening white solid which assayed at 99.7 weight % purity by HPLC and GC, indicating 97% overall yield from PET. Analysis of the filtrate indicated that it contained 2.0% of the expected yield of DMT and small amounts (<0.5% each) of methyl hydroxyethyl terephthalate (MHET) and dimethyl isophthalate (DMI).

### Example 64: Depolymerization of PET With 2-Ethylhexanol Using 2 mol % Potassium Acetate Catalyst With a 1.25:1 Ratio of 2-EH Alcohol to PET

Pelletized bottle-flake waste PET (25.00 g; 0.1276 mol) and potassium acetate (0.251 g; 0.0026 mol; 0.02 equivalents) were slurried in 2-ethylhexanol (31.3 g; 0.2399 mol; 1.88 equivalents) in a 250-mL 3-neck round-bottom flask with an overhead stirrer, a Dean-Stark trap with a condenser, and a nitrogen inlet. The flask was heated to 185 °C internal and held for 5 h (1 h after full dissolution of the PET). The flask was cooled to 60 °C and filtered. It filtered quickly and the filtrate was a flowable liquid. The filtrate analyzed at an acid by-product content of 1.86% by HPLC area% with an acid number of 1.03. The theoretical content of the filtrate was 2.26 mmol total terephthalate per gram.

### Example 65: Transesterification of PET 2-Ethylhexanolysis Mixture (2.0 mol % Potassium Acetate Catalyst; 1.25:1 2-EH alcohol to PET) with 12 equivalents methanol using sodium methoxide catalyst

PET 2-ethylhexanolysis mixture prepared as in Example 64 (2.26 mmol/g; 25.00 g; 0.0565 mol) was slurried in 27.5 mL of methanol (0.678 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (491 μL; 0.0021 mol; 0.038 equivalents; 0.30 equivalents active catalyst based on acid number) was added. The mixture was stirred at 250 rpm at 50 °C and precipitation with an attendant exotherm was noted at 5 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 9.86 g of DMT as a glistening white solid which assayed at 98.9 weight % purity by HPLC and GC, indicating 89% overall yield from PET. Analysis of the filtrate indicated that it contained 2.6% of the expected yield of DMT and small amounts (<0.5% each) of methyl hydroxyethyl terephthalate (MHET) and dimethyl isophthalate (DMI).

Example 66: Depolymerization of PET With 2-Ethylhexanol Using 2 mol % Potassium Acetate Catalyst With a 1:1 Ratio of 2-EH Alcohol to PET

Pelletized bottle-flake waste PET (25.00 g; 0.1276 mol) and potassium acetate (0.251 g; 0.0026 mol; 0.02 equivalents) were slurried in 2-ethylhexanol (25 g; 0.1920 mol; 1.50 equivalents) in a 250-mL 3-neck round-bottom flask with an overhead stirrer, a Dean-Stark trap with a condenser, and a nitrogen inlet. The flask was heated to 185 °C internal and held for 6 h (1 h after full dissolution of the PET). The flask was cooled to 60 °C and filtered. It filtered quickly and the filtrate was a flowable liquid at temperature but a little thick at ambient temperature. The filtrate analyzed at an acid by-product content of 1.88% by HPLC area% with an acid number of 0.48. The theoretical content of the filtrate was 2.54 mmol total terephthalate per gram.

### Example 67: Transesterification of PET 2-Ethylhexanolysis Mixture (2.0 mol % Potassium Acetate Catalyst; 1:1 2-EH Alcohol to PET) With 12 Equivalents Methanol Using Sodium Methoxide Catalyst

PET 2-ethylhexanolysis mixture prepared as in Example 66 (2.54 mmol/g; 25.00 g; 0.0635 mol) was slurried in 30.9 mL of methanol (0.762 mol; 12.0 equiv) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2°C) 25% sodium methoxide in methanol (479 μL; 0.0021 mol; 0.033 equivalents; 0.30 equivalents active catalyst based on acid number) was added. The mixture was stirred at 250 rpm at 50 °C and precipitation with an attendant exotherm was noted at 3.5 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 11.22 g of DMT as a glistening white solid which assayed at 94.3 weight % purity by HPLC and GC, indicating 86% overall yield from PET. Analysis of the filtrate indicated that it contained 3.5% of the expected yield of DMT and small amounts (<0.5% each) of methyl hydroxyethyl terephthalate (MHET) and dimethyl isophthalate (DMI).

### Example 68: Depolymerization of PET With 2-Ethylhexanol Using 2.5 mol % Potassium Acetate Catalyst Prepared in Situ

2-Ethylhexanol (30.6 g; 0.2349 mol; 2.26 equivalents), acetic acid (0.156 mL; 0.0027 mmol; 0.026 equivalents) and potassium hydroxide (45.9% aqueous solution; 0.318 g; 0.0026 mol; 0.025 equivalents) were combined in a 250-mL 3-neck round-bottom flask with an overhead stirrer, a Dean-Stark trap with a condenser, and a nitrogen inlet. The mixture was stirred for 5 minutes and pelletized bottle-flake waste PET (98.1% PET; 20.39 g; 0.1041 mol) was added. The flask was heated to 185 °C internal and held for 5 h (1 h after full dissolution of the PET). The filtrate analyzed at an acid by-product content of 2.40% by HPLC area% and the theoretical content of the filtrate was 2.02 mmol total terephthalate per gram.

### Example 69: Transesterification of PET 2-Ethylhexanolysis Mixture (2.5 mol % in Situ Potassium Acetate Catalyst) With 12 Equivalents Methanol Using Sodium Methoxide Catalyst

PET 2-ethylhexanolysis mixture prepared as in Example 68 (2.02 mmol/g; 25.00 g; 0.0505 mol) was slurried in 24.6 mL of methanol (0.606 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (577 μL; 0.0025 mol; 0.05 equivalents) was added. The mixture was stirred at 250 rpm at 50 °C and precipitation with an attendant exotherm was noted at 5.5 minutes. After heating for a total of 60 minutes at 50 °C the heating bath was then removed, and the mixture was allowed to cool to ambient temperature over 30 minutes and then stirred at ambient temperature for two hours. The resulting precipitate was filtered, washed with methanol, and air-dried to afford 8.54 g of DMT as a glistening white solid which assayed at 99.8 weight % purity by HPLC and GC, indicating 87% overall yield from PET. Analysis of the filtrate indicated that it contained 4.0% of the expected yield of DMT and small amounts (<0.5% each) of methyl hydroxyethyl terephthalate (MHET) and dimethyl isophthalate (DMI).

### Example 70: Preparation of 5 mol % Sodium 2-Ethylhexylate

2-Ethylhexanol (50 g) and 50% sodium hydroxide (2.672 g; 0.0334 mol) were combined in a 250-mL 3-necked rb equipped with a magnetic stir bar, a thermowell, and a Dean-Stark separator topped with an air condenser with a nitrogen/vacuum inlet. The mixture was heated in a 120 °C oil bath and vacuum was applied and the pressure was slowly decreased from 200 mm Hg to full vacuum (ca. 21 mm Hg). An initial water-2-ethanolhexanol azeotropic mixture was collected in the Dean-Stark trap followed by about 3 mL of only 2-ethylhexanol. The heat was removed and the mixture was cooled to ambient temperature and the vacuum was release with nitrogen. 2-Ethylhexanol was added to the mixture to afford a total weight of 50.73 g to afford a 10% clear colorless solution of sodium 2-ethylhexylate in 2-ethylhexanol. The density was measured as 0.82 g/mL.

### Example 71: Transesterification of PET 2-Ethylhexanolysis Mixture With 12 Equiv Methanol Using 5 mol % Sodium 2-Ethylhexylate Catalyst

PET 2-ethylhexanolysis mixture prepared as in Example 16 (2.02 mmol/g; 25.00 g; 0.0505 mol) was slurried in 24.6 mL of methanol (0.606 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 10% sodium 2-ethylhexylate in 2-ethylhexanol prepared above (4.69 mL; 0.0025 mol; 0.05 equiv) was added. The mixture was stirred at 250 rpm at 50 °C and precipitation with an attendant exotherm was noted at 7.5 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.29 g of DMT as a glistening white solid which assayed at 99.8 weight % purity by HPLC and GC, indicating 84% overall yield from PET. Analysis of the filtrate indicated that it contained 3.2% of the expected yield of DMT and small amounts (<0.5% each) of methyl hydroxyethyl terephthalate (MHET) and dimethyl isophthalate (DMI).

### Example 72: Transesterification of PET 2-Ethylhexanolysis Mixture (2-EH:PET 1.5:1) With 12 Equivalents Methanol Using Potassium Carbonate Catalyst

PET 2-ethylhexanolysis mixture prepared as in Example 16 (2.06 mmol/g; 25.0 g; 0.052 mol) was slurried in 25.1 mL of methanol (0.620 mol; 12.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) potassium carbonate (355 mg; 0.0047 mol; 0.047 equivalents; 0.040 equivalents active catalyst based on acid number) was added. The mixture was stirred at 250 rpm at 50 °C and precipitation with an attendant exotherm was noted at 10 minutes. After heating for a total of 60 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 8.52 g of DMT as a glistening white solid which assayed at 99.5 weight % purity by HPLC and GC, indicating 85% overall yield from PET. Analysis of the filtrate indicated that it contained 3.9% of the expected yield of DMT and small amounts (<0.5% each) of methyl hydroxyethyl terephthalate (MHET) and dimethyl isophthalate (DMI).

### Example 73: Transesterification of PET 2-Ethylhexanolysis Mixture (2-EH:PET 1.5:1) With 9 Equiv Methanol Using Sodium Methoxide Catalyst

PET 2-ethylhexanolysis mixture prepared as in Example 16 (2.06 mmol/g; 25.0 g; 0.052 mol) was slurried in 18.8 mL of methanol (0.465 mol; 9.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) 25% sodium methoxide in methanol (668 μL; 0.0029 mol; 0.057 equivalents; 0.050 equivalents active catalyst based on acid number) was added. The mixture was stirred at 250 rpm at 50 °C and precipitation with an attendant exotherm was noted at 5 minutes. After heating for a total of 70 minutes at 50 °C the heating bath was then removed, and the mixture was allowed to cool to ambient temperature over 30 minutes and then stirred at ambient temperature for one hour. The resulting precipitate was filtered, washed with methanol, and air-dried to afford 9.47 g of DMT as a glistening white solid which assayed at 91.3 weight % purity by HPLC and GC, indicating 86% overall yield from PET. Analysis of the filtrate indicated that it contained 1.6% of the expected yield of DMT and small amounts (<0.5% each) of methyl hydroxyethyl terephthalate (MHET) and dimethyl isophthalate (DMI).

### Example 74: Transesterification of PET 2-Ethylhexanolysis Mixture (2-EH:PET 1.5:1) With 9 Equivalents Methanol Using Potassium Carbonate Catalyst

PET 2-ethylhexanolysis mixture prepared as in Example 16 (2.03 mmol/g; 25.0 g; 0.0508 mol) was slurried in 18.51 mL of methanol (0.457 mol; 9.0 equivalents) in a 300-mL 3-neck round-bottom flask with an overhead stirrer, a thermocouple, and an air condenser with a nitrogen inlet. The flask was placed into an oil bath set at 57 °C, and once the reaction mixture equilibrated at 50 °C (+/−2 °C) potassium carbonate (379 mg; 0.0054 mol; 0.054 equivalents; 0.050 equivalents active catalyst based on acid number) was added. The mixture was stirred at 250 rpm at 50 °C and precipitation with an attendant exotherm was noted at 15.5 minutes. After heating for a total of 60 minutes at 50 °C the heating bath was then removed, and the mixture was allowed to cool to ambient temperature over 20 minutes and then stirred at ambient temperature for one hour. The resulting precipitate was filtered, washed with methanol, and air-dried to afford 8.15 g of DMT as a glistening white solid which assayed at 99.5 weight % purity by HPLC and GC, indicating 80% overall yield from PET. Analysis of the filtrate indicated that it contained 13.8% of the expected yield of DMT and small amounts (<0.5% each) of methyl hydroxyethyl terephthalate (MHET) and dimethyl isophthalate (DMI).

### Example 75: Depolymerization of Pet With 2-Ethylhexanol at a 1.5:1 Ratio

Pelletized bottle-flake waste PET (98.1% PET; 202.0 g; 1.03 mol) and potassium carbonate (3.99 g; 0.029 mol; 0.028 equivalents) were slurried in 2-ethylhexanol (303 g; 2.327 mol; 2.26 equivalents) in a 1L 3-neck round-bottom flask with an overhead stirrer, a Dean-Stark trap with a condenser, and a nitrogen inlet. The flask was heated to 185 °C internal and held for 1 h after full dissolution of the PET. The mixture was cooled to 60 °C and filtered, and the filtrate (479.66 g) was bottled. The residual solid was washed with methanol and dried to afford 12.22 g of insoluble residue. The theoretical content of the filtrate was 2.03 mmol total terephthalate per gram.

### Example 76: Transesterification of PET 2-Ethylhexanolysis Mixture (2-EH:PET 1.5:1) With Methanol Using Sodium Methoxide Catalyst and Evaporative Cooling

PET 2-ethylhexanolysis mixture prepared as in Example 16 (2.03 mmol/g; 212.00 g; 0.4295 mol) was slurried in 313 mL of methanol (7.73 mol; 18.0 equivalents) in a 1 L jacketed reactor with an overhead stirrer, a thermocouple, sequential water and dry ice condensers with a nitrogen inlet. The flask was heated to an internal temperature of 50 °C (+/−2 °C) and 25% sodium methoxide in methanol (5.89 mL; 0.026 mol; 0.06 equivalents) was added. The mixture was stirred at 50 °C for 1 h (precipitate noted at 7.5 minutes), the heating was stopped, and the mixture was evaporatively cooled to ambient temperature by reducing the pressure to about 300 mm Hg for 39 minutes. Methanol that was not condensed and directly returned to the reactor (25 mL) was added back to the mixture and the mixture then stirred at ambient temperature for one hour. The resulting precipitate was filtered, washed with methanol, and dried in a vacuum oven at 75 °C for 2 h to afford 80.36 g of DMT as a glistening white solid which assayed at 92.7 weight % purity by HPLC and GC, indicating 89% overall yield from PET. Analysis of the filtrate indicated that it contained 0.3% of the expected yield of DMT and small amounts (<0.5% each) of methyl hydroxyethyl terephthalate (MHET) and dimethyl isophthalate (DMI).

### Example 77: Depolymerization of Pet With 2-ethylhexanol at a 1.5:1 Ratio With Centrifugation

Pelletized waste carpet PET (87% PET, 25.00 g; 0.113 mol) and potassium acetate (278 mg; 0.0028 mol; 0.025 equiv) were slurried in 2-ethylhexanol (32.6 g; 0.251 mol; 2.21 equiv) in a 250-mL 3-neck round-bottom flask with a magnetic stir bar, a Dear-Stark trap with condenser, and a nitrogen inlet. The flask was heated to 185 °C and stirred for 3.5 h (PET dissolution noted at 2 h). The mixture was then allowed to cool to 60 °C and methanol (29.0 g; 0.905 mol; 8.0 equiv) was added and the resulting mixture was allowed to cool to ambient temperature. The resulting brown slurry was transferred to centrifuge tubes and centrifuged for five minutes at 3000G to afford liquid and a dense pellet. The liquid was decanted and methanol was added equally to the tubes (total 14.5 g; 0.452 mol; 4.0 equiv). The mixture was vortexed to suspend the solids and then centrifuged for five minutes at 3000 G. The resulting liquid was decanted to leave behind a dense brown pellet. The total weight of the pellet was 4.26 g and contained about 3% of the total terephthalate by HPLC analysis. The centrate and wash were combined to total 92.18 g.

### Example 78: Transesterification of PET 2-Ethylhexanolysis Mixture (2-EH:PET 1.5:1) With Methanol From Centrifuged Mixture

PET 2-ethylhexanolysis mixture prepared in Example 77 (92.18 g; 0.113 mol maximum) was added to a 500-mL 3-neck round-bottom flask with an overhead stirrer, and a thermocouple. The flask was heated to an internal temperature of 50 °C (+/−2 °C) and 25% sodium methoxide in methanol (1.29 mL; 0.0057 mol; 0.05 equivalents) was added. The mixture was stirred at 50 °C for 1 h (precipitate noted at 8.5 minutes), the heating was stopped, and the mixture was allowed to cool to ambient temperature over about 30 min, then held at ambient temperature for 1 hour. The resulting precipitate was filtered, washed with methanol, and air-dried to afford 17.27 g of DMT as a glistening white solid which assayed at >99.9 weight % purity by HPLC and GC, indicating 79% overall yield from PET. Analysis of the filtrate indicated that it contained 4.9% of the expected yield of DMT and small amounts (<0.5% each) of methyl hydroxyethyl terephthalate (MHET) and dimethyl isophthalate (DMI).

1. A process for preparing dimethyl terephthalate, the process comprising:
a. treating a poly(C₂-C₄ alkylene) terephthalate with a C₄-C14 alkanol in the presence of an esterification or transesterification catalyst at a temperature sufficient to depolymerize at least a portion of the poly(C₂-C4 alkylene terephthalate), to afford a first reaction mixture product comprising a di-C4-C14 alkyl terephthalate;
followed by
b. treatment with a transesterification catalyst in the presence of methanol, to provide a second reaction mixture product comprising dimethyl terephthalate, a C₄-C₁₄ alcohol, and ethylene glycol, followed by
c. isolation of the dimethyl terephthalate.

2. The process of claim 1, wherein step a. is conducted at a temperature of about 100 °C to about 250 °C.

3. The process of claim 1, further comprising the step of cooling the first reaction mixture product to a temperature of below about 100 °C; or further comprising the step of cooling the first reaction mixture product to a temperature of about 20 °C to about 100 °C.

4. The process of claim 3, further comprising the step of removing insoluble materials present in the first reaction mixture product by filtration, centrifugation, or decantation.

5. The process of claim 1, wherein the poly(C₂-C₄ alklene)terephthalate is poly(ethylene terephthalate).

6. The process of claim 1, wherein the C₄-C₁₄ alcohol is chosen from n-butanol, isobutanol, hexanol, 2-ethylhexanol, n-octanol, decanol, dodecanol, tetradecanol, and mixtures thereof.

7. The process of claim 1 or 6, wherein the C₄-C14 alcohol is chosen from n-butanol and 2-ethylhexanol.

8. The process of claim 1, wherein the esterification or transesterification catalyst in step a. is chosen from Lewis acids and Lewis bases; or wherein the esterification or transesterification catalyst in step a. is chosen from alkali metal C₁-C₁₄ alkoxides, alkali metal carbonates, alkali metal bicarbonates, wherein the alkali metal is chosen from lithium, sodium, potassium, or cesium; or wherein the esterification or transesterification catalyst in step a. is chosen from alkali metal acetates, transition metal acetates, and titanium C₁-C₆ alkoxides; or wherein the esterification or transesterification catalyst in step a. is chosen from potassium carbonate, potassium bicarbonate, potassium acetate, titanium tetra(isopropoxide), and monobutyltin tris(2-ethylhexanoate).

9. The process of claim 1, wherein the esterification or transesterification catalyst is chosen from tin oxalate, monobutyltin oxide, and monobutyltin tris(2-ethylhexanoate).

10. The process of claim 1, wherein the transesterification catalyst in step b. is chosen from alkali metal C₁-C₁₄ alkoxides, alkali metal hydroxides, and alkali metal carbonates; or wherein the transesterification catalyst in step b. is chosen from potassium hydroxide, sodium hydroxide, sodium methoxide, potassium methoxide, sodium 2-ethylhexylate and potassium carbonate.

11. The process of claim 1, further comprising subjecting the second reaction mixture product, after the isolation of dimethyl terephthalate in c. to distillation and/or extraction to afford purified C₄-C₁₄ alkanol and purified ethylene glycol.

12. A process for preparing dimethyl terephthalate, the process comprising:
a. treating a waste plastic stream comprising poly(ethylene terephthalate) with a C₄-C₁₄ alkanol in the presence of an esterification or transesterification catalyst at a temperature sufficient to depolymerize at least a portion of the poly(ethylene alkylene terephthalate), to afford a first reaction mixture product comprising a di-C₄-C₁₄ alkyl terephthalate; followed by
b. treatment with a transesterification catalyst in the presence of methanol, to provide a second reaction mixture product comprising dimethyl terephthalate, a C₄-C₁₄ alcohol, and ethylene glycol, followed by
c. isolation of the dimethyl terephthalate.

13. The process of claim 12, wherein step a. is conducted at a temperature of about 100 °C to about 250 °C.

14. The process of claim 12, wherein the first reaction mixture product is cooled to a temperature of about 20 °C to about 100 °C.

15. The process of claim 12, 13, or 14, further comprising the step of removing insoluble materials present in the first reaction mixture product by filtration, centrifugation, or decantation.

16. The process of claim 12, wherein the C₄-C₁₄ alcohol is chosen from n-butanol and 2-ethylhexanol.

17. The process of claim 12, wherein the esterification or transesterification catalyst in step a. is chosen from alkali metal alkoxides, alkali metal carbonates, alkali metal bicarbonates, wherein the alkali metal is chosen from lithium, sodium, potassium, or cesium; or wherein the esterification or transesterification catalyst in step a. is chosen from alkali metal acetates, transition metal acetates, and titanium C₁-C₆ alkoxides; or wherein the esterification or transesterification catalyst in step a. is chosen from tin oxalate, monobutyltin oxide, and monobutyltin tris(2-ethylhexanoate), potassium carbonate, potassium bicarbonate, potassium acetate, titanium tetra(isopropoxide), and monobutyltin tris(2-ethylhexanoate).

18. The process of claim 12, wherein the transesterification catalyst in step b. is chosen from alkali metal C₁-C₁₄ alkoxides, alkali metal hydroxides and alkali metal carbonates; or wherein the transesterification catalyst in step b. is chosen from potassium hydroxide, sodium hydroxide, sodium methoxide, potassium methoxide, sodium 2-ethylhexylate, and potassium carbonate.

19. The process of claim 12, further comprising subjecting the second reaction mixture product, after the isolation of dimethyl terephthalate in c. to distillation and/or extraction to afford purified C₄-C14 alkanol and purified ethylene glycol.
